(19) United States
(12) Patent Application Publication
(10) Pub. No.: US 2005/0271625 A1
(43) Pub. Date: Dec. 8, 2005

(54) RAAV-neprilysin compositions and methods of use

(76) Inventors: Kevin R. Nash, Gainesville, FL (US); Corinna Burger, Gainesville, FL (US)

Correspondence Address: HAYNES AND BOONE, LLP, 901 MAIN STREET, SUITE 3100, DALLAS, TX, 75202, US

(21) Appl. No.: 11/070,627

(22) Filed: Mar. 2, 2005

Related U.S. Application Data
(60) Provisional application No. 60/549,399, filed on Mar. 2, 2004.

(51) Int. Cl. A61K 48/00; C07H 21/04; C12N 15/861

(52) U.S. Cl. 424/93.2; 435/456; 435/325

(57) ABSTRACT

Disclosed are methods for the use of neprilysin-encoding polynucleotides in the creation of transformed host cells and transgenic animals. In particular, the use of recombinant adeno-associated viral (rAAV) vector compositions comprising polynucleotide sequences that express one or more biologically-active mammalian neprilysin polypeptides is described. Also disclosed are medicaments and methods for the treatment and amelioration of symptoms of a variety of conditions and neprilysin deficiencies in an animal, including, for example, Alzheimer's disease, and related disorders, as well as neurological and musculoskeletal disorders, including for example, diseases caused by the accumulation of β-amyloid protein in the cells and tissues of affected animals.

The present application claims priority to U.S. Provisional Patent Application Ser. No. 60/549,399 filed Mar. 2, 2004, the entire contents of which is specifically incorporated herein by reference in its entirety.

The United States government has certain rights in the present invention pursuant to Grant Number 2906082-03 from the National Institutes of Health.

### 1.0 BACKGROUND OF THE INVENTION

### 1.1 FIELD OF THE INVENTION

The present invention relates generally to the fields of molecular biology and virology, and in particular, to methods for using recombinant adeno-associated virus (rAAV) compositions that express nucleic acid segments encoding therapeutic gene products in the treatment of complex human disorders. In certain embodiments, the invention concerns the use of rAAV vectors that express a biologically-functional neprilysin peptide, polypeptide, or protein in a variety of investigative, diagnostic and therapeutic regimens, including the treatment of mammalian disorders and diseases, and particularly those involving the nervous and musculoskeletal systems, including, for example, neurodegeneration, memory loss, neurological impairment, and Alzheimer's disease. Methods and compositions are also provided herein for preparing rAAV vector-based neprilysin medicaments for use in viral-based gene therapies.

### 2.0 SUMMARY OF THE INVENTION

The present invention overcomes limitations inherent in the prior art by providing novel rAAV-based genetic constructs that encode one or more mammalian zinc metallopeptidases, and particularly those of the neprilysin family, for the prophylaxis, treatment and/or amelioration of symptoms of one or more mammalian diseases, disorders or dysfunctions that result from, or are exacerabated by, a deficit in, or a deficiency of, biologically-active neprilysin polypeptide activity. In particular, the invention provides genetic constructs encoding one or more mammalian neprilysin polypeptides, for use in the treatment of such conditions as β-amyloid (Aβ) protein accumulation, Alzheimer's disease, and other conditions that manifest from a deficiency or absence of physiologically-normal levels of neprilysin polypeptides, or from an abundance, or accumulation of β-amyloid protein.

The invention also provides compositions and methods for preventing, treating or ameliorating the symptoms of a neprilysin protein deficiency in a mammal, and particularly for treating or reducing the severity or extent of deficiency in a human manifesting one or more of the disorders linked to a deficiency of biologically-active neprilysin polypeptides. In a general sense, the method involves administration of at least a first rAAV-based genetic construct that encodes one or more neprilysin peptides, polypeptides, or proteins in a pharmaceutically-acceptable vehicle to the animal in an amount and for a period of time sufficient to treat or ameliorate the deficiency in the animal suspected of suffering from such a disorder. Exemplary neprilysin polypeptides useful in the practice of the invention include, but are not limited to peptides, polypeptides and proteins that have neprylisin activity, and that are substantially identical in primary amino acid sequence to any one of the sequences disclosed in SEQ ID NO:1, SEQ ID NO:2; SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and to biologically-functional equivalents, or derivatives thereof. Additional exemplary neprilysin peptides, proteins, and polypeptides useful in the practice of the include, but are not limited to those the comprise, consist essentially of, or consist of, an amino acid sequence encoding mammalian neprilysin, and particularly those sequences as disclosed in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO: 10, and to biologically-functional equivalents, or derivatives thereof.

2.1 RAAV-Neprilysin Vector Compositions

In a first embodiment, the invention provides an rAAV vector comprising a polypeptide that comprises at least a first nucleic acid segment that encodes a neprilysin peptide or polypeptide, and in particular, a mammalian neprilysin polypeptide, or a biologically-active fragment thereof, operably linked to at least a first promoter capable of expressing the nucleic acid segment in a suitable host cell transformed with such a vector. In preferred embodiments, the nucleic acid segment encodes a mammalian, and in particular, a human neprilysin polypeptide, such as for example, a polypeptide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, or a biologically-active fragment or variant thereof.

Alternatively, the therapeutic constructs of the invention may encompass nucleic acid segments that encode neprilysin polypeptides of any mammalian origin, such as for example nucleic acids, peptides, and polypeptides of murine, primate, ovine, porcine, bovine, equine, epine, caprine, canine, feline, and/or lupine origin, or may encompass modified or site-specifically mutagenized nucleic acid segments that were initially obtained from one or more mammalian species, and genetically modified to be expressed in human cells such that their neprilysin activity is retained.

In other preferred embodiments, the preferred nucleic acid segments for use in the practice of the present invention, encodes a mammalian, and in particular, a human neprilysin polypeptide or a biologically active fragment or variant thereof.

The polynucleotides comprised in the vectors and viral particles of the present invention preferably comprise at least a first constitutive or inducible promoter operably linked to a neprilysin-encoding nucleic acid segment as described herein. Such promoters may be homologous or heterologous promoters, and may be operatively positioned upstream of the nucleic acid segment encoding the neprilysin polypeptide, such that the expression of the neprilysin-encoding segment is under the control of the promoter. The construct may comprise a single promoter, or alternatively, two or more promoters may be used to facilitate expression of the neprilysin-encoding DNA sequence. Exemplary promoters useful in the practice of the invention include, but are in no way limited to, those promoter sequences that are operable in mammalian, and in particular, human host cells, tissues, and organs, such as for example, a CMV promoter, a β-actin promoter, a hybrid CMV promoter, a hybrid β-actin promoter, an EF1 promoter, a U1a promoter, a U1b promoter, a Tet-inducible promoter or a VP16-LexA promoter. In illustrative embodiments, a polynucleotide encoding a therapeutic polypeptide was placed under the control of the chicken β-actin (CBA) promoter and used to produce therapeutically-effective levels of the encoded neprilysin polypeptide when suitable host cells were transformed with the genetic construct, and the DNA encoding the neprilysin polypeptide was expressed in such cells.

The polynucleotides comprised in the vectors and viral particles of the present invention may also further optionally comprise one or more native, synthetic, homologous, heterologous, or hybrid enhancer or 5′ regulatory elements, for example, a natural enhancer, such as the CMV enhancer, or alternatively, a synthetic enhancer. Cell- or tissue-specific enhancers, including for example, those that increase expression of operably linked gene sequences are also contemplated to be particularly useful in the practice of the invention. Such enhancers may include, but are not limited to, muscle-specific enhancers, hippocampal-specific enhanvers, brain-specific enhancers, and such like.

The polynucleotides and nucleic acid segments comprised within the vectors and viral particles of the present invention may also further optionally comprise one or more intron sequences. In such instances, the intron sequence(s) will preferably be mammalian in origin, and more preferably, human in origin.

The DNA sequences, nucleic acid segments, and polynucleotides comprised within a vector, virion, viral particle, host cell, or composition of the present invention may also further optionally comprise one or more native, synthetic, homologous, heterologous, or hybrid post-transcriptional or 3′ regulatory elements operably positioned relative to the neprilysin-encoding nucleic acid segments disclosed herein to provide greater expression, greater stability, and/or enhanced translation of the encoded polypeptides. One such example is the woodchuck hepatitis virus post-transcriptional regulatory element (WPRE), operably positioned downstream of the neprilysin gene. Use of elements such as these in such circumstances is well-known to those of skill in the molecular biological arts.

In illustrative embodiments, the invention concerns administration of one or more biologically-active neprilysin proteins, peptides, or polypeptides that comprise an at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100, or more contiguous amino acid sequence from the polypeptide and peptide sequences disclosed hereinbelow, and particularly those polypeptides as recited in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Likewise, in additional illustrative embodiments, the invention concerns administration of one or more biologically-active neprilysin proteins, peptides or polypeptides that are encoded by a nucleic acid segment that comprises, consists essentially of, or consists of at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200, 250, 300, 350, 400, 450, 500, 550, 600, 650, 700, 750, or even 800 or more contiguous nucleic acid residues from the nucleic acid segments disclosed hereinbelow, and particularly those DNA sequences that encode any one or more mammalian neprilysin proteins, including for example, those that are recited in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10.

Exemplary adeno-associated viral vector constructs and polynucleotides of the present invention include those that comprise, consist essentially of, or consist of at least a first nucleic acid segment that encodes a peptide or polypeptide that is at least about 75%, at least about 80%, at least about 85%, at least about 90%, at least about 95%, at least about 96%, at least about 97%, at least about 98%, or at least about 99% identical to the sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the peptide or polypeptide has neprilysin activity when expressed in selected mammalian cells and/or tissues. In certain embodiments, the viral vector constructs and polynucleotides of the present invention will preferably include those vectors and polynucleotides that comprise, consist essentially of, or consist of at least a first nucleic acid segment that encodes a peptide or polypeptide that is at least about 82%, at least about 84%, at least about 86%, at least about 88%, at least about 92%, or at least about 94% identical to one or more of the sequences disclosed in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10. Such constructs will preferably encode one or more biologically-active peptides or polypeptides that have neprilysin activity when expressed in selected mammalian cells and/or tissues and in human cells and/or tissues in particular.

Exemplary polynucleotides of the present invention also include those sequences that comprise, consist essentially of, or consist of at least a first nucleic acid segment that is at least about 75%, at least about 80%, at least about 85%, at least about 90%, at least about 95%, at least about 96%, at least about 97%, at least about 98%, or at least about 99% identical to a nucleic acid sequence that encodes any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the peptide or polypeptide encoded by the nucleic acid segment has neprilysin activity when expressed in selected mammalian cells and/or tissues.

2.2 rAAV Viral Particles and Virions, and Host Cells Comprising them

Other aspects of the invention concern rAAV particles and virions that comprise the rAAV-neprilysin vectors of the present invention, pluralities of such particles and virions, as well as pharmaceutical compositions and host cells that comprise one or more of the rAAV-neprilysin vectors disclosed herein, such as for example pharmaceutical formulations of the rAAV-neprilysin vectors or virions intended for administration to a mammal through suitable means, such as, by intramuscular, intravenous, or direct injection to selected cells, tissues, or organs of the mammal, for example, one or more regions of the brain of the selected mammal. Typically, such compositions will be formulated with pharmaceutically-acceptable excipients, buffers, diluents, adjuvants, or carriers, as described hereinbelow, and may further comprise one or more liposomes, lipids, lipid complexes, microspheres, microparticles, nanospheres, or nanoparticle formulations to facilitate administration to the selected organs, tissues, and cells for which therapy is desired.

Further aspects of the invention include mammalian host cells, and pluralities thereof that comprise one or more of the adeno-associated viral vectors, virions, or viral particles as disclosed herein. Particularly preferred cells are human host cells, and in particular, human brain cells and tissues.

2.3 Therapeutic Kits and Pharmaceutical Compositions

Therapeutic kits for treating or ameliorating the symptoms of a condition resulting from a neprilysin deficiency in a mammal are also part of the present invention. Exemplary kits are those that preferably comprise one or more of the disclosed AAV-neprilysin vector constructs, virions, or pharmaceutical compositions described herein, and instructions for using the kit. The use of such kits in methods of treatment of neprilysin deficiency is particularly contemplated in the treatment of β-amyloid protein accumulation in tissues or cells of an affected mammal.

Another important aspect of the present invention concerns use of the disclosed vectors, virions, compositions, and host cells described herein in the preparation of medicaments for treating or ameliorating the symptoms of neprilysin deficiency in a mammal, and in particular, a human. The use of such compositions in the preparation of medicaments and in methods for the treatment of neurological and/or central nervous system defects, including for example, conditions resulting from an accumulation of β-amyloid protein, such as for example in Alzheimer's disease, generally involve administration to a mammal, and particularly to a human in need thereof, one or more of the disclosed viral vectors, virionss, host cells, or compositions comprising one or more of them, in an amount and for a time sufficient to treat or ameliorate the symptoms of such a deficiency in the affected mammal. The methods may also encompass prophylactic treatment of animals suspected of having such conditions, or administration of such compositions to those animals at risk for developing such conditions either following diagnosis, or prior to the onset of symptoms. Such symptoms may include, but are not limited to, neurological deficit, memory loss, or Alzheimer's disease, or any other medical condition that may result from a reduction, deficiency, or absence of biologically-functional neprilysin polypeptide activity.

Another aspect of the invention concerns compositions that comprise one or more of the disclosed adeno-associated viral vectors, virions, viral particles, and host cells as described herein. Pharmaceutical compositions comprising such are particularly contemplated to be useful in therapy, and particularly in the preparation of medicaments for treating affected mammals, and humans in particular.

2.4 Therapeutic Methods

The invention also provides methods for delivering therapeutically-effective amounts of a neprilysin polypeptide to a mammal in need thereof. Such methods generally comprise at least the step of providing or administering to such a mammal, one or more of the neprilysin compositions disclosed herein. For example, the method may involve providing to such a mammal, one or more of the rAAV vectors, virions, viral particles, host cells, or pharmaceutical compositions as described herein. Preferably such providing or such administration will be in an amount and for a time effective to provide a therapeutically-effective amount of one or more of the neprilysin polypeptides disclosed herein to selected cells, tissues, or organs of the mammal, and in particular, therapeutically-effective levels to the cells of the mammalian brain. Such methods may include systemic injection(s) of the therapeuticum, or may even involve direct or indirect administration, injection, or introduction of the therapeutic compositions to particular cells, tissues, or organs of the mammal.

For example, the therapeutic composition may be provided to mammal by direct injection to the tissues of the brain or to the intracerebroventricular space, or to the hippocampal region of the brain.

The invention also provides methods of treating, ameliorating the symptoms, and reducing the severity of neprilysin deficiency in an animal. These methods generally involve at least the step of providing to an animal in need thereof, one or more of the rAAV neprilysin vector compositions disclosed herein in an amount and for a time effective to treat β-amyloid protein accumulation, or to treat a dysfunction resulting from such accumulation, or resulting from an underexpresison or absence of sufficient biologically-active neprilysin polypeptide in the animal. As described above, such methods may involve systemic injection(s) of the therapeuticum, or may even involve direct or indirect administration, injection, or introduction of the therapeutic compositions to particular cells, tissues, or organs of the animal.

The invention further concerns the use of the adeno-associated viral vectors, virions, viral particles, host cells, and/or the pharmaceutical compositions disclosed herein in the manufacture of a medicament for treating neprilysin deficiency, beta Amyloid protein accumulation, or Alzheimer's disease or other memory-loss or age-related mental dysfunction in a mammal. This use may involve systemic or localized injection, infection, or administration to one or more cells, tissues, or organs of the mammal. Such use is particularly contemplated in humans that have, are suspected of having, or at risk for developing one or more neurological dysfunctions such as Alzheimer's disease.

### 3.0 BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1 shows rAAV construct expressing the Neprilysin gene under the control of the chicken β-actin (CBA) promoter. Note signal peptide in NEP-s.

FIG. 2A, FIG. 2B, FIG. 2C and FIG. 2D show distribution of NEP 1.5 mo after injections of rAAV vectors. Brain sections were immunostained for NEP with an antibody recognizing both rodent and human NEP. Sections are shown from uninjected control hippocampus (FIG. 2A), hippocampus injected with rAAV-NEP-n (FIG. 2B), hippocampus injected with rAAV-NEP-s (FIG. 2C) and hippocampus contralateral (FIG. 2D) to that injected with NEP-s. (40× magnification).

FIG. 3A, FIG. 3B, FIG. 3C, FIG. 3D, FIG. 3E and FIG. 3F show measurement of Aβ and Thioflavine S loads in mice injected with rAAV vectors. Mice were either untreated (Control) or injected with one of three rAAV vectors (GFP, NEP-s or NEP-n). Brain sections were collected 1.5 mo after the injections and stained for either Aβ (FIG. 3A, FIG. 3B and FIG. 3C) or Thioflavine S (FIG. 3D, FIG. 3E and FIG. 3F) and the stained area quantified by image analysis. Ipsilateral refers to the side injected with AAV and contralateral refers to the side opposite that injected. *=P<0.05 vs. Control group except for FIG. 3C where P<0.05 vs. GFP injected mice (Fischer's PLSD test after ANOVA; Statview software by SAS).

FIG. 4A, FIG. 4B, FIG. 4C, FIG. 4D, FIG. 4E and FIG. 4F show Aβ immunohistochemistry after rAAV injections. Brain sections were immunostained for Aβ with a polyclonal antiserum. Sections are shown from the right (EPSI) and left (CONTRA) sides of an uninjected control mouse (Uninjected: FIG. 4A and FIG. 4B), a mouse injected with rAAV-NEP-s (FIG. 4C and FIG. 4D), a mouse injected with rAAV-GFP (FIG. 4E) and a mouse injected with rAAV-NEP-n (FIG. 4F). The box in FIG. 4E approximates field imaged for quantification. (40× magnification).

FIG. 5A, FIG. 5B, FIG. 5C, FIG. 5D, FIG. 5E and FIG. 5F show Thioflavine S fluorescence after rAAV injections. Brain sections were stained with Thioflavine S and visualized with epifluorescence. Sections are shown from the injected ispilateral side (IPSI, FIG. 5A, FIG. 5C and FIG. 5E) and from the side contralateral to the injection (CONTRA; FIG. 5B, FIG. 5D and FIG. 5F). FIG. 5A and FIG. 5B are from an uninjected control mouse (Uninj.), FIG. 5C and FIG. 5D are from a mouse injected with rAAV-NEP-s, and FIG. 5E and FIG. 5F are from a mouse injected with rAAV NEP-n. (40× magnification).

FIG. 6A, FIG. 6B, FIG. 6C and FIG. 6D show sections from transgenic mouse hippocampi that have been injected with NEPsp (secreted neprilysin) (FIG. 6A), intact NEP full length neprilysin) (FIG. 6B), and an untreated mouse all stained with antisera against neprilysin (FIG. 6C). There is a general increase in background staining throughout much of the hippocampus in mice given the NEPsp construct (FIG. 6A). This differs considerably from the highly localized increase in pyramidal cell staining obtained with intact NEP (FIG. 6B). The endogenous murine neprilysin staining is clearly less than the AAV injected mice (FIG. 6C). FIG. 6D shows a mouse injected with a vector containing only green fluorescent proteins, showing the cells capable of transfection by the AAV vector in mouse hippocampus. This relative distribution of increased neprilysin immunoreactivity is precisely what was expected from the two vectors used, indicating that the signal sequence guiding secretion worked as predicted.

FIG. 7 is a graph showing AAV-NEP vectors reduce Aβ load in anterior cortex.

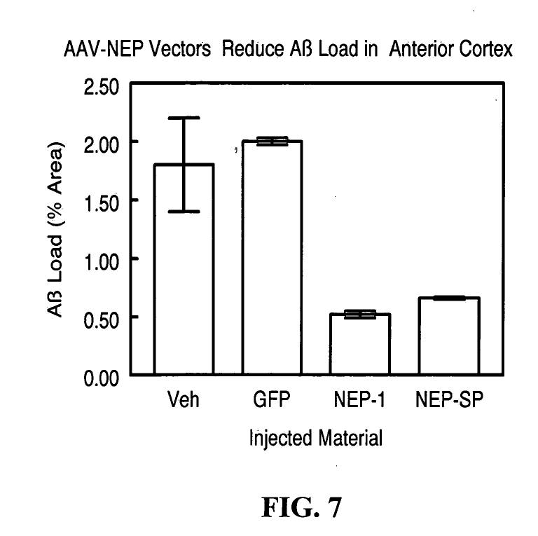

FIG. 8 is a graph showing AAV-NEP vectors reduce Aβ load in hippocampus.

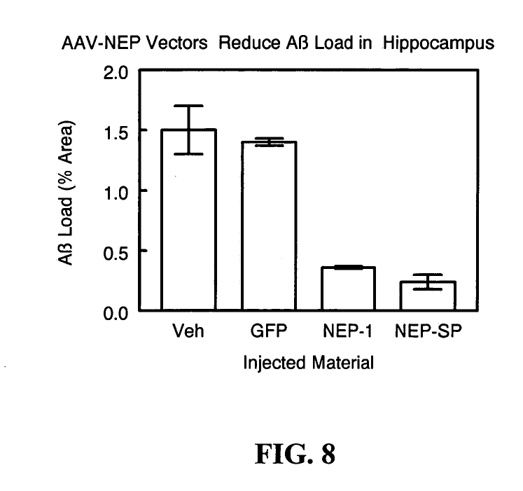

FIG. 9 is a graph showing AAV-NEP vectors reduce Thioflavin S load in frontal cortex.

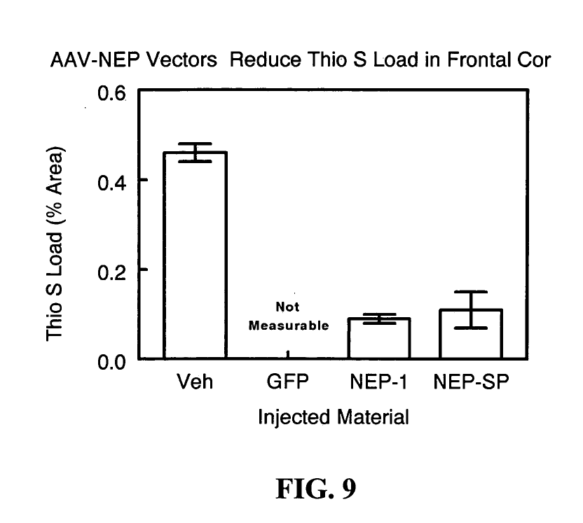

FIG. 10 is a graph showing AAV-NEP vectors reduce Thioflavin S load in hippocampus.

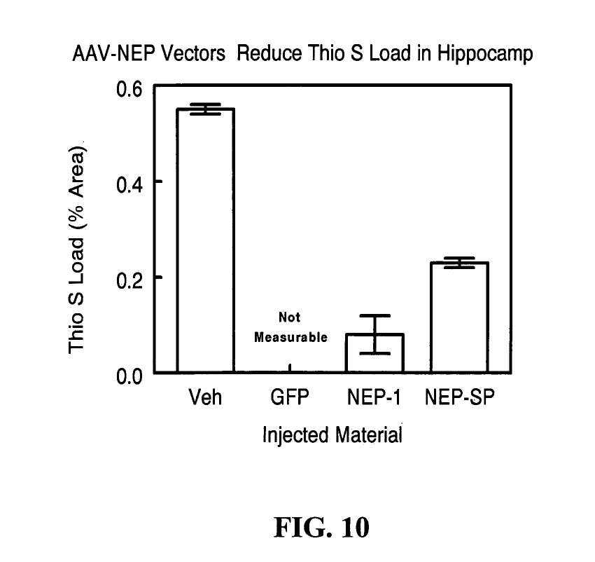

### 4.0 DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

4.1 Adeno-Associated Virus

Adeno-associated virus-2 (AAV) is a human parvovirus that can be propagated both as a lytic virus and as a provirus (Cukor et al., 1984; Hoggan et al., 1972). The viral genome consists of linear single-stranded DNA (Rose et al., 1969), 4679 bases long (Srivastava et al., 1983), flanked by inverted terminal repeats of 145 bases (Lusby et al., 1982). For lytic growth AAV requires co-infection with a helper virus. Either adenovirus (Atchinson et al., 1965; Hoggan, 1965; Parks et al., 1967) or herpes simplex (Buller et al., 1981) can supply helper function. Without helper, there is no evidence of AAV-specific replication or gene expression (Rose and Koczot, 1972; Carter et al., 1983). When no helper is available, AAV can persist as an integrated provirus (Hoggan, 1965; Bems et al., 1975; Handa et al., 1977; Cheung et al., 1980; Bems et al., 1982).

Integration apparently involves recombination between AAV termini and host sequences and most of the AAV sequences remain intact in the provirus. The ability of AAV to integrate into host DNA is apparently an inherent strategy for insuring the survival of AAV sequences in the absence of the helper virus. When cells carrying an AAV provirus are subsequently superinfected with a helper, the integrated AAV genome is rescued and a productive lytic cycle occurs (Hoggan, 1965).

AAV sequences cloned into prokaryotic plasmids are infectious (Samulski et al., 1982). For example, when the wild type AAV/pBR322 plasmid, pSM620, is transfected into human cells in the presence of adenovirus, the AAV sequences are rescued from the plasmid and a normal AAV lytic cycle ensues (Samulski et al., 1982). This renders it possible to modify the AAV sequences in the recombinant plasmid and, then, to grow a viral stock of the mutant by transfecting the plasmid into human cells (Samulski et al., 1983; Hermonat et al., 1984). AAV contains at least three phenotypically distinct regions (Hermonat et al., 1984). The rep region codes for one or more proteins that are required for DNA replication and for rescue from the recombinant plasmid, while the cap and lip regions appear to code for AAV capsid proteins and mutants within these regions are capable of DNA replication (Hermonat et al., 1984). It has been shown that the AAV termini are required for DNA replication (Samnulski et al., 1983).

Laughlin et al. (1983) have described the construction of two E. coli hybrid plasmids, each of which contains the entire DNA genome of AAV, and the transfection of the recombinant DNAs into human cell lines in the presence of helper adenovirus to successfully rescue and replicate the AAV genome (See also Tratschin et al., 1984a; 1984b).

Adeno-associated virus (AAV) is particularly attractive for gene transfer because it does not induce any pathogenic response and can integrate into the host cellular chromosome (Kotin et al., 1990). The AAV terminal repeats (TRs) are the only essential cis-components for the chromosomal integration (Muzyczka and McLaughin, 1988). These TRs are reported to have promoter activity (Flotte et al., 1993). They may promote efficient gene transfer from the cytoplasm to the nucleus or increase the stability of plasmid DNA and enable longer-lasting gene expression (Bartlett and Samulski, 1998). Studies using recombinant plasmid DNAs containing AAV TRs have attracted considerable interest. AAV-based plasmids have been shown to drive higher and longer transgene expression than the identical plasmids lacking the TRs of AAV in most cell types (Philip et al., 1994; Shafron et al., 1998; Wang et al., 1998).

There are several factors that prompted researchers to study the possibility of using rAAV as an expression vector. One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering the antisense constructs of the present invention.

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response. AAV therefore, represents an ideal candidate for delivery of the neprilysin-encoding polynucleotides of the present invention.

4.2 Production of rAAV Vectors

Traditional protocols to produce rAAV vectors have generally been based on a three-component system. One component of this system is a proviral plasmid encoding the recombinant DNA to be packaged as rAAV. This recombinant DNA is located between 145 base pair (bp) AAV-2 inverted terminal repeats (ITRs) that are the minimal cis acting AAV-2 sequences that direct replication and packaging of the vector. A second component of the system is a plasmid encoding the AAV-2 genes, rep and cap. The AAV-2 rep gene encodes four Rep proteins (Rep 78, 68, 52 and 40) that act in trans to replicate the rAAV genome, resolve replicative intermediates, and then package single-stranded rAAV genomes. The AAV-2 cap gene encodes the three structural proteins (VP1, VP2, and VP3) that comprise the virus capsid. Because AAV-2 does not proficiently replicate on its own, the third component of a rAAV packaging system is a set of helper functions from another DNA virus. These helper functions create a cellular environment in which rAAV replication and packaging can efficiently occur. The helper functions provided by adenovirus (Ad) have almost exclusively been used to produce rAAV and are encoded by the genes E1a, E1b, E2a, E4orf6, and VA RNA. While the first two components of the system are generally introduced into cells in which replication and packaging is to occur by transfection, ad helper functions are introduced by superinfection with wild type Ad virus.

The traditional rAAV production techniques are limited in their ability to produce large quantities of vector because of inherent inefficiencies in transfection. Serious difficulties are also encountered when the scale of transfection is increased. The requirement for wild type Ad may also reduce the amount of rAAV produced since Ad may compete for cellular and viral substrates that are required for viral replication but are present only in limiting amounts. Another problem encountered in traditional production protocols is that superinfection with Ad requires development of effective procedures for purification of Ad from the rAAV produced. While these purification processes are generally successful at eliminating Ad contamination of rAAV preparations, they also reduce rAAV titers. Stringent assays for Ad contamination of rAAV are also necessary.

To produce rAAV, a double co-transfection procedure is used to introduce a rAAV transfer vector plasmid together with pDG (Grimm et al., 1998) AAV helper plasmid carrying the AAV rep and cap genes, as well as Ad helper genes required for rAAV replication and packaging at a 1:1 molar ratio. Plasmid DNA used in the transfection is purified by a conventional alkaline lysis/CsCl gradient protocol. The transfection is carried out as follows: 293 cells are split 1:2 the day prior to the experiment, so that, when transfected, the cell confluence is about 75-80%. Ten 15-cm plates are transfected as one batch. To make CaPO₄ precipitate 0.7 mg of pDG are mixed with 180 μg of rAAV transfer vector plasmid in a total volume of 12.5 ml of 0.25 M CaCl₂. The old media is removed from the cells and the formation of the CaPO₄-precipitate is initiated by adding 12/5 ml of 2×HBS pH 7.05 (pre-warmed at 37° C.) to the DNA-CaCl₂ solution. The DNA is incubated for 1 min; and transferring the mixture into pre-warmed 200 ml of DMEM-10% FBS then stops the formation of the precipitate. Twenty two ml of the media is immediately dispensed into each plate and cells are incubated at 37° C. for 48 hrs. The CaPO₄-precipitate is allowed to stay on the cells during the whole incubation period without compromising cell viability. Forty-eight hr post-transfection cells are harvested by centrifugation at 1,140×g for 10 min. Cells are then lysed in 15 ml of 0.15 M MaCl, 50 mM tris HCl pH 8.5 by 3 freeze/thaw cycles in dry ice-ethanol and 37° C. baths. Benzonase (Nycomed Pharma A/S, pure grade) is added to the mixture (50 U/ml, final concentration) and the lysate is incubated for 30 min at 37° C. The lysate is clarified by centrifugation at 3,700 g for 20 min and the virus-containing supernatant is further purified using a discontinuous density gradient.

The typical discontinuous step gradient is formed by underlayering and displacing the less dense cell lysate with Iodixanol, 5,5″[(2-hydroxi-1-3-propanediyl)-bis(acetylamino)]bis [N,N′bi, (2,3dihydroxypropyl-2-4,6-triiodo-1,3-enzenecarboxamide], prepared using a 60% (wt./vol.) sterile solution of OptiPrep (Nycomed). Specifically, 15 ml of the clarified lysate are transferred into Quick-Seal Ultra-Clear 25×89 mm centrifuge tube (Beckman) using a syringe equipped with 1/27×89 mm spinal needle. Care is taken to avoid bubbles, which would interfere with subsequent filling and sealing of the tube. A variable speed peristaltic pump, Model EP-1 (Bio-Rad), is used to underlay in order: 9 ml of 15% iodixanol and 1 M NaCl in PBS-MK buffer containing Phenol Red (2.5 μl of a 0.5% stock solution per ml of the iodixanol solution); 5 ml of 40% iodixanol in PBS-MK buffer; and finally, 5 ml of 60% iodixanol in PBS-MK buffer containing Phenol Red (0.1 μl/l). Tubes are sealed and centrifuged in a Type 70 Ti rotor (Beckman) at 350,000×g for 1 hr at 18° C. Four ml of the clear 40% step is aspirated after puncturing the tube on the side with a syringe equipped with an 18-gauge needle with the bevel uppermost. The iodixanol fraction is further purified using conventional Heparin agarose affinity chromatography.

For chromatography, typically, a pre-packed 2.5 ml Heparin agarose Type I column (Sigma) is equilibrated with 20 ml of PBS-MK under gravity. The rAAV iodixanol fraction is then applied to the pre-equilibrated column, and the column is washed with 10 ml of PBS-MK. rAAV is eluted with the same buffer containing 1M NaCl. After applying the elution buffer, the first 2 ml of the eluant are discarded, and the virus is collected in the subsequent 3.5 ml of elution buffer.

Virus is then concentrated and desalted by centrifugation through the BIOMAX 100 K filter (Millipore) according to the manufacturer instructions. The high salt buffer is changed by repeatedly diluting the concentrated virus with the Lactated Ringer's solution and repeating the centrifugation.

To characterize the quality of the virus, two assays are used to titer both physical and infectious rAAV particles. A conventional dot-blot assay or quantitative competitive PCR™ (QR PCR™) assay are used to determine physical particle titers. Infectious titers are determined by infectious center assay (ICA) and fluorescent cell assay (FCA), which scores for expression of GFP.

QC PCR™ method is based on competitive co-amplified of a specific target sequence with internal standard plasmid of known concentration in on reaction tube. It provides precise and fast quantitation of viral particles. The internal standard must hare primer recognition sites with the specific template. Both the specific template and the internal standard must be PCR™—amplified with the same efficiency and it must be possible to analyze the PCR™—amplified products separately. The easiest way to distinguish between the template and the internal standard is to incorporate a size difference in the two products. This can be achieved, for example, by constructing standards having the same sequence as the specific target but containing a deletion. Quantitation is then performed by comparing the PCR™ signal of the specific template with the PCR™ signal obtained with known concentrations of the competitor (the internal standard).

The purified viral stock is first treated with DNAseI to digest any contaminating unpackaged DNA. Ten μl of a purified virus stock is incubated with 10 U of DNA se I (Boehringer) in a 100 μl reaction mixture, containing 50 mM Tris HCl, pH 7.5, 10 mM MgCl₂ for 1 hr at 37° C. At the end of the reaction, 10 μl of 10× Protinase K buffer (10 mM Tris HCl, pH 8.0, 10 mM EDTA, 1% SDS final concentration) is added, followed by the addition of 1 μl of Proteinase K (18.6 mg/ml, Boehringer). The mixture is incubated at 37° C. for one hour. Viral DNA is purified by phenol/chloroform extraction (twice), followed by chloroform extraction and ethanol precipitation using 10 μg of glycogen as a carrier. The DNA pellet is dissolved in 100 μl of water. QC PCR™ reaction mixtures each contain 1 μl of the diluted viral DNA and two-fold serial dilutions of the internal standard plasmid DNA, such as pdl-GFP. The most reliable range of standard DNA was found to be between 1 and 100 pg. And aliquot of each reaction is the analyzed by 2% agarose gel electrophoresis, until two PCR™ products are resolved. The analog image of the ethidium bromide stained gel is digitized using and ImageStore 7500 system (UVP). The densities of the target and competitor bands in each lane are measured using the ZERO-Dscan Image Analysis System, version 1.0 (Scanalytics) and their ratios are plotted as a function of the standard DNA concentration. A ratio of 1, at which the number of viral DNA molecules equals the number of competitor DNA molecules is used to determine the DNA concentration of the virus stock.

A modification of the previously published protocol (McLaughlin et al., 1988) is used to measure the ability of the virus to infect C12 cells, unpackage, and replicate. Briefly, C2 cells containing integrated wtAAV rep and cap genes (Clark et al., 1995), are plated in a 96-well dish at about 75% confluence and infected with Ad5 at the M.O.I of 20. One μl of serially diluted rAAV-sCNTF is visually scored using a fluorescence microscope. High sensitivity CHROMA filter #41012 High Q FITC LP is used to monitor the fluorescence. To calculate the titer by hybridization, cells are harvested and processed essentially ad described earlier (McLaughlin et al., 1988).

4.3 Promoters and Enhancers

Recombinant AAV vectors form important aspects of the present invention. The term “expression vector or construct” means any type of genetic construct containing a nucleic acid in which part or all of the nucleic acid encoding sequence is capable of being transcribed. In preferred embodiments, expression only includes transcription of the nucleic acid, for example, to generate a neprilysin polypeptide product from a transcribed gene.

Particularly useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

In preferred embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a neprilysin-encoding gene in its natural environment. Such promoters may include promoters normally associated with other genes, and/or promoters isolated from any other bacterial, viral, eukaryotic, or mammalian cell.

Naturally, it will be important to employ a promoter that effectively directs the expression of the neprilysin-encoding DNA segment in the cell type, organism, or even animal, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high-level expression of the introduced DNA segment.

At least one module in a promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter, such as a CMV or an HSV promoter. In certain aspects of the invention, tetracycline controlled promoters are contemplated.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters that are well known in the art to achieve expression of a transgene is contemplated as well, provided that the levels of expression are sufficient for a given purpose. Tables 1 and 2 below list several elements/promoters that may be employed, in the context of the present invention, to regulate the expression of the present neprilysin-encoding constructs. This list is not intended to be exhaustive of all the possible elements involved in the promotion of transgene expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
PROMOTER AND ENHANCER ELEMENTS

| PROMOTER/ENHANCER | REFERENCES |
|---|---|
| Immunoglobulin Heavy Chain | Banerji et al., 1983; Gilles et al., 1983; Grosschedl and |
| | Baltimore, 1985; Atchinson and Perry, 1986, 1987; Imler |
| | et al., 1987; Weinberger et al., 1984; Kiledjian et al., |
| | 1988; Porton et al.; 1990 |
| Immunoglobulin Light Chain | Queen and Baltimore, 1983; Picard and Schaffner, 1984 |
| T-Cell Receptor | Luria et al., 1987; Winoto and Baltimore, 1989; Redondo |
| | et al.; 1990 |
| HLA DQ a and DQ β | Sullivan and Peterlin, 1987 |
| β-Interferon | Goodbourn et al., 1986; Fujita et al., 1987; Goodbourn |
| | and Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick and Benfield, 1989; Johnson |
| | et al., 1989 |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987; Tronche et al., 1989, 1990 |
| α-Fetoprotein | Godbout et al., 1988; Campere and Tilghman, 1989 |
| t-Globin | Bodine and Ley, 1987; Perez-Stable and Constantini, 1990 |
| β-Globin | Trudel and Constantini, 1987 |
| e-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau etal., 1981; Sleigh and |
| | Lockett, 1985; Firak and Subramanian, 1986; Herr and |
| | Clarke, 1986; Imbra and Karin, 1986; Kadesch and Berg, |
| | 1986; Wang and Calame, 1986; Ondek et al., 1987; Kuhl |
| | et al., 1987; Schaffner et al., 1988 |
| Polyoma | Swartzendruber and Lehman, 1975; Vasseur et al., 1980; |
| | Katinka et al., 1980, 1981; Tyndell et al., 1981; Dandolo |
| | et al., 1983; de Villiers et al., 1984; Hen et al., 1986; |
| | Satake et al., 1988; Campbell and Villarreal., 1988 |
| Retroviruses | Kriegler and Botchan, 1982, 1983; Levinson et al., 1982; |
| | Kriegler et al., 1983, 1984a, b, 1988; Bosze et al., 1986; |
| | Miksicek et al., 1986; Celander and Haseltine, 1987; |
| | Thiesen et al., 1988; Celander et al., 1988; Chol et al., |
| | 1988; Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; Hirochika |
| | etal., 1987; Stephens and Hentschel, 1987 |
| Hepatitis B Virus | Bulla and Siddiqui, 1986; Jameel and Siddiqui, 1986; |
| | Shaul and Ben-Levy, 1987; Spandau and Lee, 1988; |
| | Vannice and Levinson, 1988 |
| Human Immunodeficiency Virus | Muesing et al., 1987; Hauber and Cullan, 1988; |
| | Jakobovits et al., 1988; Feng and Holland, 1988; Takebe |
| | et al., 1988; Rosen et al., 1988; Berkhout et al., 1989; |
| | Laspia et al., 1989; Sharp and Marciniak, 1989; Braddock |
| | et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

TABLE 2
INDUCIBLE ELEMENTS

| ELEMENT | INDUCER | REFERENCES |
|---|---|---|
| MT II | Phorbol Ester (TFA) | Palmiter et al., 1982; |
| | Heavy metals | Haslinger and Karin, 1985; |
| | | Searle et al., 1985; Stuart |
| | | et al., 1985; Imagawa et |
| | | al., 1987, Karin et al., |
| | | 1987; Angel et al., 1987b; |
| | | McNeall et al., 1989 |
| MMTV (mouse | Glucocorticoids | Huang et al., 1981; Lee et |
| mammary | | al., 1981; Majors and Varmus, |
| tumor virus) | | 1983; Chandler et al., 1983; |
| | | Lee et al., 1984; Ponta et |
| | | al., 1985; Sakai et al., 1988 |
| β-Interferon | poly(rI)x | Tavernier et al., 1983 |
| | poly(rc) | |
| Adenovirus | Ela | Imperiale and Nevins, 1984 |
| 5 E2 | | |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX | Interferon, | |
| Gene | Newcastle | |
| | Disease Virus | |
| GRP78 Gene | A23187 | Resendez et al., 1988 |
| α-2- | IL-6 | Kunz et al., 1989 |
| Macroglobulin | | |
| Vimentin | Serum | Rittling et al., 1989 |
| MHC Class | Interferon | Blanar et al., 1989 |
| I Gene | | |
| H-2κb | | |
| HSP70 | Ela, SV40 Large | Taylor et al., 1989; Taylor |
| | T Antigen | and Kingston, 1990a, b |
| Proliferin | Phorbol Ester-TPA | Mordacq and Linzer, 1989 |
| Tumor Necrosis | FMA | Hensel et al., 1989 |
| Factor | | |
| Thyroid | Thyroid Hormone | Chatterjee et al., 1989 |
| Stimulating | | |
| Hormone a Gene | | |

As used herein, the terms “engineered” and “recombinant” cells are intended to refer to a cell into which an exogenous DNA segment, such as DNA segment that leads to the transcription of a neprilysin, or a ribozyme specific for such a polypeptide product, has been introduced. Therefore, engineered cells are distinguishable from naturally occurring cells, which do not contain a recombinantly introduced exogenous DNA segment. Engineered cells are thus cells having DNA segment introduced through the hand of man.

To express a neprilysin gene in accordance with the present invention one would prepare an rAAV expression vector that comprises a neprilysin-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. Particularly preferred recombinant vector constucts are those that comprise an rAAV vector encoding a biologically-active neprilysin polypeptide. Such vectors are described in detail herein.

4.4 Pharmaceutical Compositions

In certain embodiments, the present invention concerns formulation of one or more of the rAAV-neprilysin compositions disclosed herein in pharmaceutically acceptable solutions for administration to a cell or an animal, either alone, or in combination with one or more other modalities of therapy.

It will also be understood that, if desired, the nucleic acid segment, RNA, DNA or PNA compositions that express a therapeutic gene product as disclosed herein may be administered in combination with other agents as well, such as, e.g., proteins or polypeptides or various pharmaceutically-active agents, including one or more systemic or topical administrations of neprilysin polypeptides. In fact, there is virtually no limit to other components that may also be included, given that the additional agents do not cause a significant adverse effect upon contact with the target cells or host tissues. The rAAV-vectored neprilysin compositions may thus be delivered along with various other agents as required in the particular instance. Such compositions may be purified from host cells or other biological sources, or alternatively may be chemically synthesized as described herein. Likewise, such compositions may further comprise substituted or derivatized RNA, DNA, or PNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the pharmaceutical compositions disclosed herein parenterally, intravenously, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety). Solutions of the active compounds as freebase or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various other ingredients as enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

4.5 Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the rAAV vector compositions of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV-neprilysin constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e., in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; Couvreur, 1988; zur Muhlen et al., 1998; Zambaux et al. 1998; Pinto-Alphandry et al., 1995 and U.S. Pat. No. 5,145,684, specifically incorporated herein by reference in its entirety).

4.6 Additional Modes of Delivery

In addition to the methods of delivery described above, the following techniques are also contemplated as alternative methods of delivering the rAAV vector compositions to a target cell or animal. Sonophoresis (i.e., ultrasound) has been used and described in U.S. Pat. No. 5,656,016 (specifically incorporated herein by reference in its entirety) as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. No. 5,770,219 and U.S. Pat. No. 5,783,208) and feedback-controlled delivery (U.S. Pat. No. 5,697,899), each specifically incorporated herein by reference in its entirety.

4.7 Therapeutic and Diagnostic Kits

The invention also encompasses one or more compositions together with one or more pharmaceutically-acceptable excipients, carriers, diluents, adjuvants, and/or other components, as may be employed in the formulation of particular rAAV-neprilysin formulations, and in the preparation of therapeutic agents for administration to a mammal, and in particularly, to a human, for one or more of the neprilysin-deficient conditions described herein. In particular, such kits may comprise one or more rAAV-neprilysin composition in combination with instructions for using the viral vector in the treatment of such disorders in a mammal, and may typically further include containers prepared for convenient commercial packaging.

As such, preferred animals for administration of the pharmaceutical compositions disclosed herein include mammals, and particularly humans. Other preferred animals include murines, bovines, equines, porcines, canines, and felines. The composition may include partially or significantly purified rAAV-neprilysin compositions, either alone, or in combination with one or more additional active ingredients, which may be obtained from natural or recombinant sources, or which may be obtainable naturally or either chemically synthesized, or alternatively produced in vitro from recombinant host cells expressing DNA segments encoding such additional active ingredients.

Therapeutic kits may also be prepared that comprise at least one of the compositions disclosed herein and instructions for using the composition as a therapeutic agent. The container means for such kits may typically comprise at least one vial, test tube, flask, bottle, syringe or other container means, into which the disclosed rAAV composition(s) may be placed, and preferably suitably aliquoted. Where a second neprilysin composition is also provided, the kit may also contain a second distinct container means into which this second composition may be placed. Alternatively, the plurality of neprilysin compositions may be prepared in a single pharmaceutical composition, and may be packaged in a single container means, such as a vial, flask, syringe, bottle, or other suitable single container means. The kits of the present invention will also typically include a means for containing the vial(s) in close confinement for commercial sale, such as, e.g., injection or blow-molded plastic containers into which the desired vial(s) are retained.

4.8 Methods of Nucleic Acid Delivery and DNA Transfection

In certain embodiments, it is contemplated that one or more of the rAAV-delivered neprilysin-encoding RNA, DNA, PNAs and/or substituted polynucleotide compositions disclosed herein will be used to transfect an appropriate host cell. Technology for introduction of rAAVs comprising one or more PNAs, RNAs, and DNAs into target host cells is well known to those of skill in the art.

Several non-viral methods for the transfer of expression constructs into cultured mammalian cells also are contemplated by the present invention for use in certain in vitro embodiments, and under conditions where the use of rAAV-mediated delivery is less desirable. These include calcium phosphate precipitation (Graham and Van Der Eb, 1973; Chen and Okayama, 1987; Rippe et al., 1990) DEAE-dextran (Gopal, 1985), electroporation (Wong and Neumann, 1982; Fromm et al., 1985; Tur-Kaspa et al., 1986; Potter et al., 1984; Suzuki et al., 1998; Vanbever et al., 1998), direct microinjection (Capecchi, 1980; Harland and Weintraub, 1985), DNA-loaded liposomes (Nicolau and Sene, 1982; Fraley et al., 1979; Takakura, 1998) and lipofectamine-DNA complexes, cell sonication (Fechheimer et al., 1987), gene bombardment using high velocity microprojectiles (Yang et al., 1990; Klein et al., 1992), and receptor-mediated transfection (Curiel et al., 1991; Wagner et al., 1992; Wu and Wu, 1987; Wu and Wu, 1988). Some of these techniques may be successfully adapted for in vivo or ex vivo use.

4.9 Expression in Animal Cells

The inventors contemplate that a polynucleotide comprising a contiguous nucleic acid sequence that encodes a therapeutic neprilysin polypeptide of the present invention may be utilized to treat one or more cellular defects in a transformed host cell. Such cells are preferably animal cells, including mammalian cells such as those obtained from a human or other primate, murine, canine, bovine, equine, epine, or porcine species. In particular, the use of such constructs for the treatment and/or amelioration of eating disorders or neurological dysfunction in a human subject suspected of suffering from such a disorder is highly contemplated. The cells may be transformed with one or more rAAV vectors comprising one or more therapeutic neprilysin genes of interest, such that the genetic construct introduced into and expressed in the host cells of the animal is sufficient to alter, reduce, ameliorate or prevent the deleterious or disease conditions either in vitro and/or in vivo.

4.10 Transgenic Animals

It is contemplated that in some instances the genome of a transgenic non-human animal of the present invention will have been altered through the stable introduction of one or more of the rAAV-delivered neprilysin polynucleotide compositions described herein, either native, synthetically modified, or mutated. As used herein, the term “transgenic animal” is intended to refer to an animal that has incorporated exogenous DNA sequences into its genome. In designing a heterologous neprilysin gene for expression in animals, sequences which interfere with the efficacy of gene expression, such as polyadenylation signals, polymerase II termination sequences, hairpins, consensus splice sites and the like are eliminated. Current advances in transgenic approaches and techniques have permitted the manipulation of a variety of animal genomes via gene addition, gene deletion, or gene modifications (Franz et al., 1997). For example, mosquitos (Fallon, 1996), trout (Ono et al., 1997), zebrafish (Caldovic and Hackett, 1995), pigs (Van Cott et al., 1997) and cows (Haskell and Bowen, 1995), are just a few of the many animals being studied by transgenics. The creation of transgenic animals that express human proteins such as a-1-antitrypsin, in sheep (Carver et al., 1993); decay accelerating factor, in pigs (Cozzi et al., 1997), and plasminogen activator, in goats (Ebert et al., 1991) has previously been demonstrated. The transgenic synthesis of human hemoglobin (U.S. Pat. No. 5,602,306) and fibrinogen (U.S. Pat. No. 5,639,940) in non-human animals have also been disclosed, each specifically incorporated herein by reference in its entirety. Further, transgenic mice and rat models have recently been described as new directions to study and treat cardiovascular diseases such as hypertension in humans (Franz et al., 1997; Pinto-Siestma and Paul, 1997).

The construction of a transgenic mouse model has recently been used to assay potential treatments for Alzheimer's disease (U.S. Pat. No. 5,720,936, specifically incorporated herein by reference in its entirety). It is contemplated in the present invention that transgenic animals contribute valuable information as models for studying the effects of neprilysin compositions on correcting genetic defects and treating a variety of disorders in an animal.

4.11 DNA Integration, RNA Expression and Inheritance

Genomic DNA may be isolated from animal cell lines or any animal parts to determine the presence of the exogenously introduced neprilysin-encoding genenetic construct through the use of one or more readily-available techniques that are well known to those skilled in the art. The presence of DNA elements introduced through the methods of this invention may be determined by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. In addition, it is not possible using PCR™ techniques to determine whether transformants have exogenous genes introduced into different sites in the genome, ie., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™ e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Whereas DNA analysis techniques may be conducted using DNA isolated from any part of an animal, RNA will only be expressed in particular cells or tissue types and hence it will be necessary to prepare RNA for analysis from these tissues. PCR™ techniques may also be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridization. These techniques are modifications of Northern blotting and will only demonstrate the presence or absence of an RNA species.

4.12 Selectable Markers

In certain embodiments of the invention, the delivery of a nucleic acid in a cell, and in particular, an rAAV construct that expresses one or more therapeutic neprilysin compositions may be identified in vitro or in vivo by including a marker in the expression construct. The marker would result in an identifiable change to the transfected cell permitting ready identification of expression. Usually the inclusion of a drug selection marker aids in cloning and in the selection of transformants, for example, neomycin, puromycin, hygromycin, DHFR, GPT, zeocin and histidinol. Alternatively, enzymes such as herpes simplex virus thymidine kinase (tk) (eukaryotic) or chloramphenicol acetyltransferase (CAT) (prokaryotic) may be employed, as well as markers such as green fluorescent protein, luciferase, and the like. Immunologic markers also can be employed. The selectable marker employed is not believed to be important, as long as it is capable of being expressed simultaneously with the nucleic acid encoding a gene product. Further examples of selectable markers are well known to one of skill in the art.

4.13 Site-Specific Mutagenesis

Site-specific mutagenesis is a technique useful in the preparation of individual peptides, or biologically functional equivalent polypeptides, through specific mutagenesis of the underlying polynucleotides that encode them. The technique, well-known to those of skill in the art, further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or change the properties of the polynucleotide itself, and/or alter the properties, activity, composition, stability, or primary sequence of the encoded polypeptide.

In certain embodiments of the present invention, the inventors contemplate the mutagenesis of the contemplated neprilysin-encoding polynucleotide sequences to alter the activity or effectiveness of such constructs in increasing or altering their therapeutic activity in a transformed host cell. Likewise in certain embodiments, the inventors contemplate the mutagenesis of such genes themselves, or of the rAAV delivery vehicle to facilitate improved regulation of the particular neprilysin polypeptide's activity, solubility, stability, expression, or efficacy in vitro and/or in vivo.

The techniques of site-specific mutagenesis are well known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation that result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing. Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Qβ Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site (Walker et al., 1992), may also be useful in the amplification of nucleic acids in the present invention.

Strand Displacement Amplification (SDA) is another method of carrying out isothermal amplification of nucleic acids that involves multiple rounds of strand displacement and synthesis, i.e., nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having a 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods are described in Great Britain Pat. Appl. No. 2 202 328, and in PCT Intl. Pat. Appl. Publ. No. PCT/US89/01025, each of which is incorporated herein by reference in its entirety, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H(RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, disclose a nucleic acid sequence amplification scheme based on the hybridization of a promoter/primer sequence to a target single-stranded DNA (“ssDNA”) followed by transcription of many RNA copies of the sequence. This scheme is not cyclic; i.e., new templates are not produced from the resultant RNA transcripts. Other amplification methods include “RACE” (Frohman, 1990), and “one-sided PCR” (Ohara et al., 1989) which are well-known to those of skill in the art.

Methods based on ligation of two (or more) oligonucleotides in the presence of nucleic acid having the sequence of the resulting “di-oligonucleotide,” thereby amplifying the di-oligonucleotide (Wu and Dean, 1996, incorporated herein by reference in its entirety), may also be used in the amplification of DNA sequences of the present invention.

4.14 Biological Functional Equivalents

Modification and changes may be made in the structure of the rAAV vector-delivered neprilysin compositions, or the polynucleotides and/or encoded neprilysin polypeptides of the present invention and still obtain a functional molecule that encodes a neprilysin polypeptide with desirable biological activity. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 3.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

TABLE 3

| AMINO ACIDS | | | CODONS | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAG | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

4.15 Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 (specifically incorporated herein by reference) reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Six basic varieties of naturally occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., 1992). Thus, the specificity of action of a ribozyme is greater than that of an antisense oligonucleotide binding the same RNA site.

The enzymatic nucleic acid molecule may be formed in a hammerhead, hairpin, a hepatitis 6 virus, group I intron or RNaseP RNA (in association with an RNA guide sequence) or Neurospora VS RNA motif. Examples of hammerhead motifs are described by Rossi et al. (1992). Examples of hairpin motifs are described by Hampel et al. (Eur. Pat. Appl. Publ. No. EP 0360257), Hampel and Tritz (1989), Hampel et al. (1990) and U.S. Pat. No. 5,631,359 (specifically incorporated herein by reference). An example of the hepatitis δ virus motif is described by Perrotta and Been (1992); an example of the RNaseP motif is described by Guerrier-Takada etal (1983); Neurospora VS RNA ribozyme motif is described by Collins (Saville and Collins, 1990; Saville and Collins, 1991; Collins and Olive, 1993); and an example of the Group I intron is described in U.S. Pat. No. 4,987,071 (specifically incorporated herein by reference). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

In certain embodiments, it may be important to produce enzymatic cleaving agents that exhibit a high degree of specificity for the RNA of a desired target, such as one of the neprilysin sequences disclosed herein. The enzymatic nucleic acid molecule is preferably targeted to a highly conserved sequence region of a target mRNA. Such enzymatic nucleic acid molecules can be delivered exogenously to specific cells as required, although in preferred embodiments the ribozymes are expressed from DNA or RNA vectors that are delivered to specific cells.

Small enzymatic nucleic acid motifs (e.g., of the hammerhead or the hairpin structure) may also be used for exogenous delivery. The simple structure of these molecules increases the ability of the enzymatic nucleic acid to invade targeted regions of the mRNA structure. Alternatively, catalytic RNA molecules can be expressed within cells from eukaryotic promoters (e.g., Scanlon et al., 1991; Kashani-Sabet et al., 1992; Dropulic et al., 1992; Weerasinghe et al., 1991; Ojwang et al., 1992; Chen et al., 1992; Sarver et al., 1990). Those skilled in the art realize that any ribozyme can be expressed in eukaryotic cells from the appropriate DNA vector. The activity of such ribozymes can be augmented by their release from the primary transcript by a second ribozyme (Int. Pat. Appl. Publ. No. WO 93/23569, and Int. Pat. Appl. Publ. No. WO 94/02595, both hereby incorporated by reference; Ohkawa et al., 1992; Taira et al., 1991; and Ventura et al., 1993).

Ribozymes may be added directly, or can be complexed with cationic lipids, lipid complexes, packaged within liposomes, or otherwise delivered to target cells. The RNA or RNA complexes can be locally administered to relevant tissues ex vivo, or in vivo through injection, aerosol inhalation, infusion pump or stent, with or without their incorporation in biopolymers.

Ribozymes may be designed as described in Int. Pat. Appl. Publ. No. WO 93/23569 and Int. Pat. Appl. Publ. No. WO 94/02595 (each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Hammerhead or hairpin ribozymes may be individually analyzed by computer folding (Jaeger et al., 1989) to assess whether the ribozyme sequences fold into the appropriate secondary structure, as described herein. Those ribozymes with unfavorable intramolecular interactions between the binding arms and the catalytic core are eliminated from consideration. Varying binding arm lengths can be chosen to optimize activity. Generally, at least 5 or so bases on each arm are able to bind to, or otherwise interact with, the target RNA.

Ribozyines of the hammerhead or hairpin motif may be designed to anneal to various sites in the mRNA message, and can be chemically synthesized. The method of synthesis used follows the procedure for normal RNA synthesis as described in Usman et al. (1987) and in Scaringe et al. (1990) and makes use of common nucleic acid protecting and coupling groups, such as dimethoxytrityl at the 5′-end, and phosphoramidites at the 3′-end. Average stepwise coupling yields are typically >98%. Hairpin ribozymes may be synthesized in two parts and annealed to reconstruct an active ribozyme (Chowrira and Burke, 1992). Ribozymes may be modified extensively to enhance stability by modification with nuclease resistant groups, for example, 2′-amino, 2′-C-allyl, 2′-flouro, 2′-o-methyl, 2′-H (for a review see e.g, Usman and Cedergren, 1992). Ribozymes may be purified by gel electrophoresis using general methods or by high-pressure liquid chromatography and resuspended in water.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Perreault et al, 1990; Pieken et al., 1991; Usman and Cedergren, 1992; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

A preferred means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells (Elroy-Stein and Moss, 1990; Gao and Huang, 1993; Lieber et al., 1993; Zhou et al., 1990). Ribozymes expressed from such promoters can function in mammalian cells (Kashani-Sabet et al., 1992; Ojwang et al., 1992; Chen et al., 1992; Yu et al., 1993; L'Huillier et al., 1992; Lisziewicz et al., 1993). Although incorporation of the present ribozyme constructs into adeno-associated viral vectors is preferred, such transcription units can be incorporated into a variety of vectors for introduction into mammalian cells, including but not restricted to, plasmid DNA vectors, other viral DNA vectors (such as adenovirus vectors), or viral RNA vectors (such as retroviral, semliki forest virus, sindbis virus vectors).

Sullivan et al. (Int. Pat. Appl. Publ. No. WO 94/02595) describes general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Ribozymes of this invention may be used to inhibit gene expression and define the role (essentially) of specified gene products in the progression of disease. In this manner, other genetic targets may be defined as important mediators of the disease. These studies lead to better treatment of the disease progression by affording the possibility of combination therapies (e.g., multiple ribozymes targeted to different genes, ribozymes coupled with known small molecule inhibitors, or intermittent treatment with combinations of ribozymes and/or other chemical or biological molecules).

4.16 Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

A, an: In accordance with long standing patent law convention, the words “a” and “an” when used in this application, including the claims, denotes “one or more.”

Expression: The combination of intracellular processes, including transcription and translation undergone by a polynucleotide such as a structural gene to synthesize the encoded peptide or polypeptide.

Promoter: a term used to generally describe the region or regions of a nucleic acid sequence that regulates transcription.

Regulatory Element: a term used to generally describe the region or regions of a nucleic acid sequence that regulates transcription. Exemplary regulatory elements include, but are not limited to, enhancers, post-transcriptional elements, transcriptional control sequences, and such like.

Structural gene: A polynucleotide, such as a gene, that is expressed to produce an encoded peptide, polypeptide, protein, ribozyme, catalytic RNA molecule, or antisense molecule.

Transformation: A process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

Transformed cell: A host cell whose nucleic acid complement has been altered by the introduction of one or more exogenous polynucleotides into that cell.

Transgenic cell: Any cell derived or regenerated from a transformed cell or derived from a transgenic cell, or from the progeny or offspring of any generation of such a transformed host cell.

Vector: A nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

The terms “substantially corresponds to,” “substantially homologous,” or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as, e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s) which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted on the basis of known consensus sequence motifs, or by other methods known to those of skill in the art.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linked” means that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, 2, 3, 4, 5, 6, 7, 8, 9, or 10 base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary oligonucleotide sequences will be greater than about 80 percent complementary (or “% exact-match”) to the corresponding mRNA target sequence to which the oligonucleotide specifically binds, and will, more preferably be greater than about 85 percent complementary to the corresponding mRNA target sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 90 percent complementary to the corresponding mRNA target sequence to which the oligonucleotide specifically binds, and may in certain embodiments be greater than about 95 percent complementary to the corresponding mRNA target sequence to which the oligonucleotide specifically binds, and even up to and including 96%, 97%, 98%, 99%, and even 100% exact match complementary to all or a portion of the target mRNA to which the designed oligonucleotide specifically binds.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

### 5.0 EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 EXAMPLE 1

### rAAV-Neprilysin Vectors in APP+PS1 Transgenic Mice

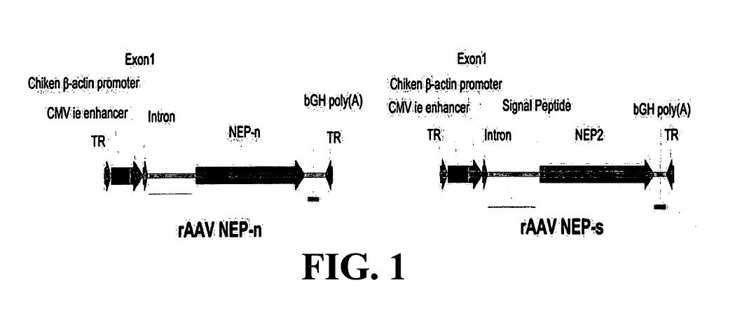

This example evaluates the effects of rAAV vectors on Aβ deposition in the brains of APP+PS1 transgenic mice. rAAV vectors were injected unilaterally into the right hippocampus and right anterior cortex of 6 mo old transgenic mice (2 μl each site). The effects were examined one month after the injections using the methods and constructs described below. Mice injected with rAAV-NEP-s (a secreted form of the neprilysin enzyme, FIG. 1), rAAV NEP-n (a native version of neprilysin), rAAV-GFP (to control for rAAV injection alone) and control untreated mice were compared.

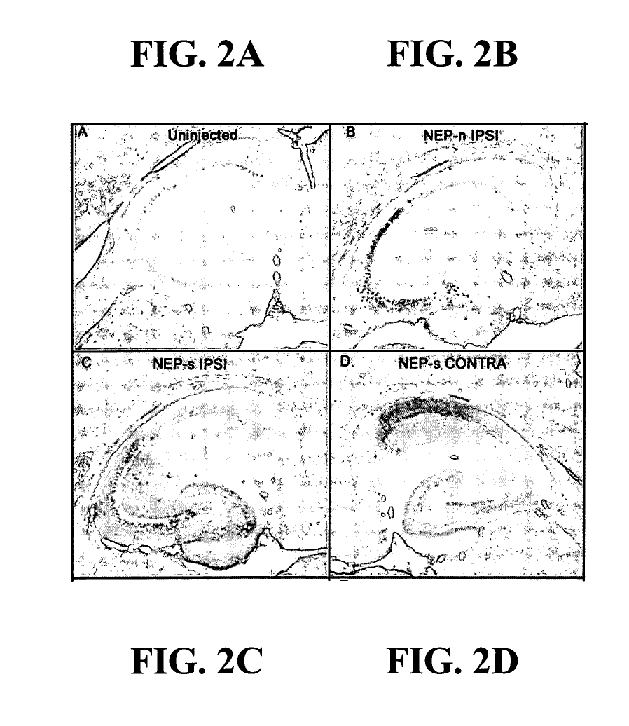

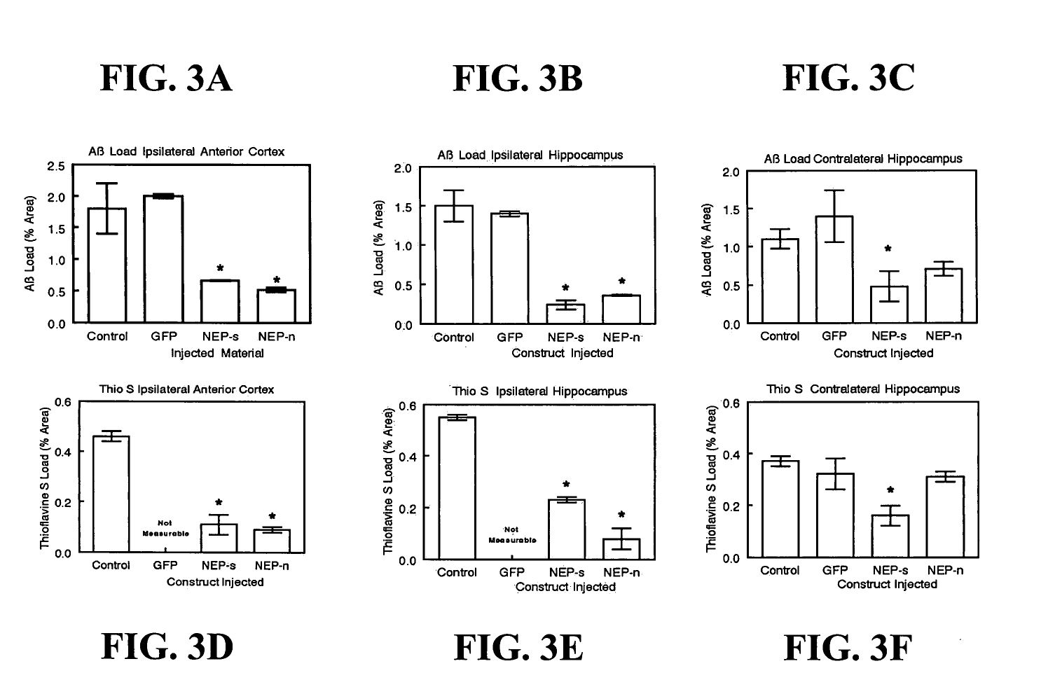

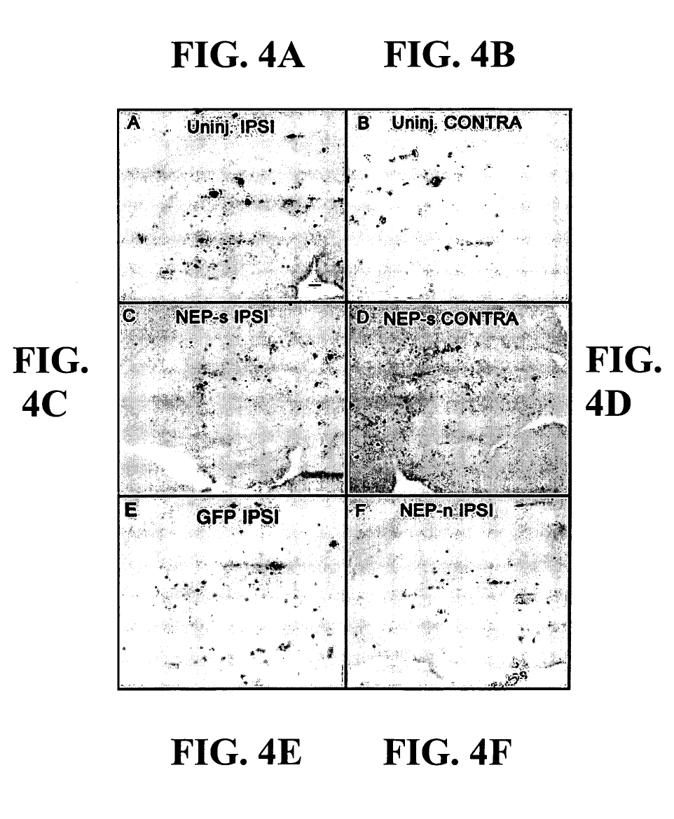

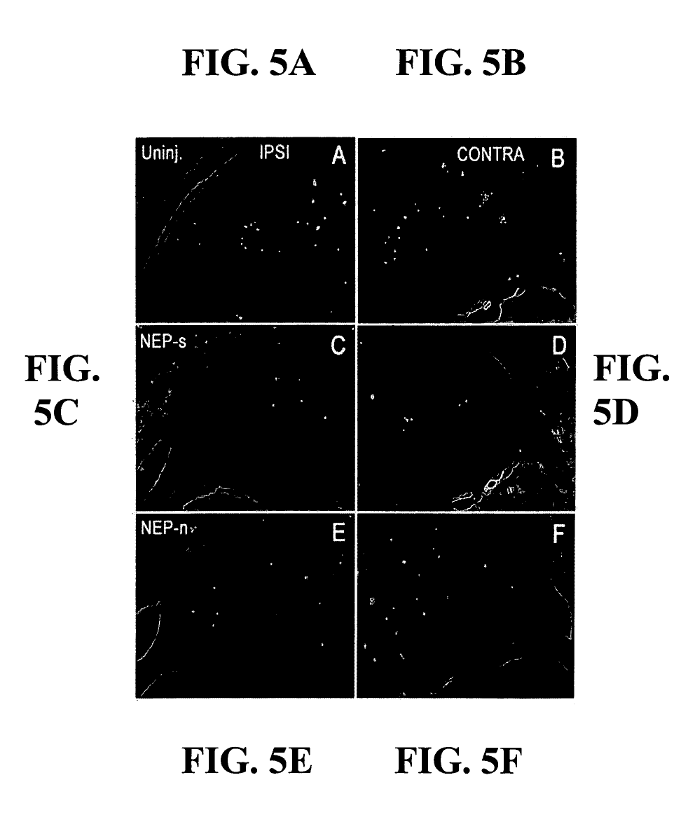

The first goal was to compare the pattern of expression of the NEP-s and NEP-n rAAV constructs. It was found that the levels of endogenous NEP in the hippocampus of transgenic mice were relatively low and were concentrated in the somatic layers of the hippocampus (FIG. 2A). These levels were increased with the injection of rAAV-NEP-n primarily in the CA3 region, not far from the site targeted by the stereotaxic coordinates near the hilus. The pattern of staining was similar to that observed for the endogenous enzyme, again concentrated in pyramidal cell layer, but more intense (FIG. 2B). In contrast, the pattern of staining with the NEP-s vector on the side ipsilateral to the injection was strikingly different (FIG. 2C). The NEP-s vector resulted in a diffuse increase in staining throughout the neuropil, with relatively little staining of neuronal somata in the pyramidal and granule cell layers. It extended beyond the CA3 regions into CA1-2 and the dentate gyrus. A similar pattern of staining was found in the hippocampus contralateral to the AAV-NEP-s injection (FIG. 2D). Here, a diffuse pattern of staining restricted to the inner two thirds of the molecular layers of the dentate gyrus and Ammon's horn was found, a distribution not unlike that of the commissural and associational fibers intrinsic to the hippocampus. In spite of the relatively brief duration of exposure to the transferred NEP genes, both NEP-s and NEP-n were able to reduce the Aβ loads at the site of injection in the anterior cortex (FIG. 3A). The NEP-s vector was able to significantly reduce the hippocampal Aβ loads on both sides of the brain (FIG. 3C, FIG. 3D, FIG. 4C and FIG. 4D), consistent with the bilateral increases of the protease after unilateral injection. NEP-n was also able to reduce Aβ loads on the side ipsilateral to the injection (FIG. 3B and FIG. 4F). Importantly, in the contralateral cerebral cortex, there was no change in Aβ load in any group, with all means within 10% of the untreated mice. This indicates the effects were not simply a result of randomly selecting mice with inherently lower Aβ loads for the rAAV treatments. A similar set of results were obtained from sections stained with Thioflavine S, which stains the fibrillar deposits within the hippocampus (FIG. 5A-5F). One problem with the rAAV-GFP group was that the cells transduced by this rAAV vector fluoresced green in the same channel as Thioflavine S. Therefore, it was not possible to measure Thioflavine S staining in rAAV GFP mice on the side ipsilateral to the injection. As for the Aβ staining, these results showed that for NEP-n and NEP-s, Thio S staining was reduced at both injection sites in anterior cortex (FIG. 4D) and hippocampus (FIG. 4E and FIG. 5E). The NEP-s reduction was significant both ipsilateral and contralateral to the injection site (FIG. 4E, FIG. 4F, FIG. 5C and FIG. 5D), consistent with the Aβ results and the distribution of the NEP staining. Note that in the hippocampus opposite the rAAV-GFP injections, the Thioflavine S measurements could be performed without interference. On this side, there was no difference between the untreated control mice and the GFP injected animals (FIG. 4F). In the contralateral anterior cortex, all four groups had similar amounts of Thioflavine S positive staining.

These results indicated that the rAAV-NEP vectors were effective in reducing Aβ deposition. Additionally, the data suggest that the NEP-s vector is secreted by the hippocampal neurons as intended, given its diffuse distribution into the molecular layers of Ammon's horn and dentate gyrus. There is even a suggestion that either there is axonal transport of the transduced gene product, or, alternatively, there is uptake by synapses projecting from the contralateral hippocampus, as there was sufficient NEP present on the side opposite the injection to significantly deplete amyloid.

### 5.2 EXAMPLE 2

### Preparation of rAAV Neprilysin Constructs

This example describes the construction of the following vectors: AAV with full length neprilysin (EC 3.4.24.11) and AAV with a truncated neprilysin from amino acid 53-743 with a secretion signal sequence engineered into the 5′ (ATGAAGTTATGGGATGTCGTGGCTGTCTGCCTGGTGCTGCTCCACACCGCGTCCG CCG). (SEQ ID NO: 11). The later construct was created for the production of a secreted soluble neprilysin.

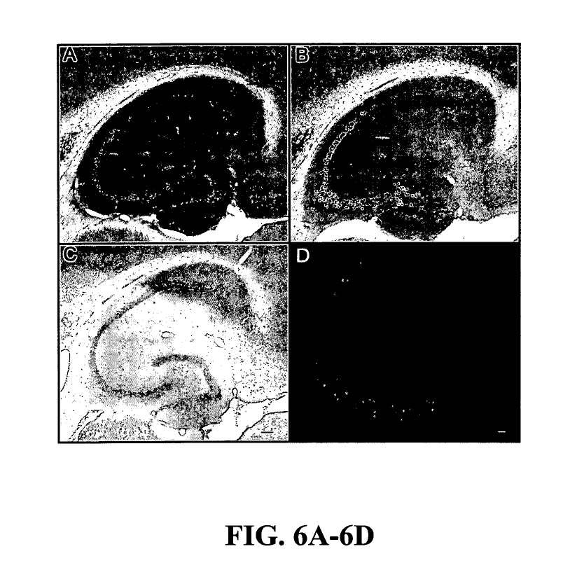

FIG. 6A, FIG. 6B, FIG. 6C and FIG. 6D show sections from transgenic mouse hippocampi that have been injected with NEPsp (secreted neprilysin) (FIG. 6A), intact NEP full length neprilysin) (FIG. 6B), and an untreated mouse all stained with antisera against neprilysin (FIG. 6C). There is a general increase in background staining throughout much of the hippocampus in mice given the NEPsp construct (FIG. 6A). This differs considerably from the highly localized increase in pyramidal cell staining obtained with intact NEP (FIG. 6B). The endogenous murine neprilysin staining is clearly less than the AAV injected mice (FIG. 6C). FIG. 6D shows a mouse injected with a vector containing only green fluorescent proteins, showing the cells capable of transfection by the AAV vector in mouse hippocampus. This relative distribution of increased neprilysin immunoreactivity is precisely what was expected from the two vectors used indicating that the signal sequence guiding secretion worked as predicted (Table 4).

TABLE 4
RESULTS OF TRANSGENIC MICE INJECTIONS

| | Hippocampus | Cerebral Cortex |
|---|---|---|
| GFP | 1.4 ± .03 | 1.98 ± .03 |
| NEP-1 | 0.36 ± .02 | 0.52 ± .03 |
| NEP-SP | 0.24 ± .06 | 0.66 ± .01 |
| CTL | 1.5 ± .16 | 1.8 ± .41 |

N = 2 for all groups.
Aβ ICC (immuno-cytochemistry) area positively stained (Aβ load).
Mean ± sem.

The Neprilysin injected animals had ˜80% lower Aβ area positively stained. ANOVAs (analysis or variants) are significant at P<0.01 and Fischers LSD show NEP groups lower than GFP or CTL groups. Congo red staining for Aβ plaques will be performed.

### 5.3 EXAMPLE 3

### Anti-Amyloid Gene Therapy for Alzheimer'S Disease

Reductions in amyloid load caused by expression of human neprilysin in trangenic mouse anterior cortex and hippocampus. Mice were injected and sacrificed one month later. The expression of neprilysin was monitored as described in the previous example. Aβ immunohisotochemistry and thioflavin S histochemistry in tissue were measured from these mice.

In both hippocampus and frontal cortex there was a substantial and statistically signficant (P<0.03 to P<0.001) reduction in amyloid deposition in mice injected with the AAV-neprilysin containing vectors compared to control mice and mice injected with AAV-GFP vectors. For Thioflavin S, the GFP (green fluorescent protein) interfered with the measurement of the yellow-green Thioflavin S fluorescence. These data strongly argue that the neprilysin expression is capable of degrading the Aβ deposits in these 8 month old APP+PS1 transgenic mice.

6.0 Exemplary Mammalian Neprilysin (Neutral Endopeptidase) Polypeptide Sequences

From GenBank Accession Number NP_{—}009220 Human (Homo sapiens) neprilysin (SEQ ID NO:1)

MGKSESQMDITDINTPKPKKKQRWTRLEISLSVLVLLLTIIAVRMIALYA
TYDDGICKSSDCIKSAARLIQNMDATTEPCRDFFKYACGGWLKRNVIPET
SSRYGNFDILRDELEVVLKDVLQEPKTEDIVAVQKAKALYRSCINESAID
SRGGEPLLKLLPDIYGWPVATENWEQKYGASWTAEKAIAQLNSKYGKKVL
INLFVGTDDKNSVNHVIHIDQPRLGLPSRDYYECTGIYKEACTAYVDFMI
SVARLIRQEERLPIDENQLALEMNKVMELEKEIANATAKPEDRNDPMLLY
NKMRLAQIQNNFSLEINGKPFSWLNFTNEIMSTVNISITNEEDVVVYAPE
YLTKLKPILTKYSARDLQNLMSWRFIMDLVSSLSRTYKESRNAFRKALYG
TTSETATWRRCANYVNGNMENAVGRLYVEAAFAGESKHVVEDLIAQIREV
FIQTLDDLTWMDAETKKRAEEKALAIKERIGYPDDIVSNDNKLNNEYLEL
NYKEDEYFENIIQNLKFSQSKQLKKLREKVDKDEWISGAAVVNAFYSSGR
NQIVFPAGILQPPFFSAQQSNSLNYGGIGMVIGHEITHGFDDNGRNFNKD
GDLVDWWTQQSASNFKEQSQCMVYQYGNFSWDLAGGQHLNGINTLGENIA
DNGGLGQAYRAYQNYIKKNGEEKLLPGLDLNHKQLEFLNFAQVWCGTYRP
EYAVNSIKTDVHSPGNFRIIGTLQNSAEFSEAFHCRKNSYMNPEKKCRVW

From GenBank Accession Number P07861 Rat (Rattus norvegicus) neprilysin (SEQ ID NO:2)

MGRSESQMDITDINAPKPKKKQRWTPLEISLSVLVLLLTIIAVTMIALYA
TYDDGICKSSDCIKSAARLIQNMDASAEPCTDFFKYACGGWLKRNVIPET
SSRYSNFDILRDELEVILKDVLQEPKTEDIVAVQKAKTLYRSCINESAID
SRGGQPLLTLLPDIYGWPVASQNWEQTYGTSWTAEKSIAQLNSKYGKKVL
INFFVGTDDKNSTQHIIHFDQPRLGLPSRDYYECTGIYKEACTAYVDFMI
SVARLIRQEQRLPIDENQLSLEMNKVMELEKEIANATTKPEDRNDPMLLY
NKMTLAKLQNNFSLEINGKPFSWSNFTNEIMSTVNINIQNEEEVVVYAPE
YLTKLKPILTKYSPRDLQNLMSWRFIMDLVSSLSRNYKESRNAFRKALYG
TTSETATWRRCANYVNGNMENAVGRLYVEAAFAGESKHVVEDLIAQIREV
FIQTLDDLTWMDAETKKKAEEKALAIKERIGYPDDIISNENKLNNEYLEL
NYKEEEYFENIIQNLKFSQSKQLKKLREKVDKDEWISGAAVVNAFYSSGR
NQIVFPAGILQPPFFSARQSNSLNYGGIGMVIGHEITHGFDDNGRNFNKD
GDLVDWWTQQSANNFKDQSQCMVYQYGNFTWDLAGGQHLNGINTLGENIA
DNGGIGQAYRAYQNYVKKNGEEKLLPGLDLNHKQLFFLNFAQVWCGTYRP
EYAVNSIKTDVHSPGNFRIIGTLQNSAEFADAFHCRKNSYMNPERKCRVW

From GenBank Accession Number Q61391 Mouse (Mus musculus) neprilysin (SEQ ID NO:3)

MGRSESQMDITDINAPKPKKKQRWTPLEISLSVLVLLXTIIAVTMTALYA
TYDDGICKSSDCIKSAARLIQNMDASVEPCTDFFKYACGGWLKRNVIPET
SSRYSNFDILRDELEVILKDVLQEPKTEDIVAVQKAKTLYRSCINESAID
SRGGQPLLKLLPDIYGWPVASDNWDQTYGTSWTAEKSIAQLNSKYGKKVL
INFFVGTDDKNSTQHIIHFDQPRLGLPSRGYYECTGIYKEACTAYVDFMI
SVARLIRQEQSLPIDENQLSLEMNKVMELEKEIANATTKPEDRNDPMLLY
NKMTLAKLQNNFSLEVNGKSFSWSNFTNEIMSTVNINIQNEEEVVVYAPE
YLTKLKPILTKYSPRDLQNLMSWRFTMDLVSSLSRNYKESRNAFRKALYG
TTSETATWRRCANYVNGNMENAVGRLYVEAAFAGESKHVVEDLIAQIREV
FIQTLDDLTWMDAETKKKAEEKALAIKERIGYPDDIISNENKLNNEYLEL
NYREDEYFENIIQNLKFSQSKQLKKLREKVDKDEWISGAAVVNAFYSSGR
NQIVFPAGILQPPFFSAQQSNSLNYGGIGMVIGHEITHGFDDNGRNFNKD
GDLVDWWTQQSANNFKDQSQCMVYQYGNFSWDLAGGQHLNGINTLGENIA
DNGGIGQAYRAYQNYVKKNGEEKLLPGLDLNHKQLFFLNFAQVWCGTYRP
EYAVNSIKTDVHSPGNFRIIGTLQNSAEFADAFHCRKNSYMNPERKCRVW

From GenBank Accession Number P08049 Rabbit (Oryctolagus cuniculus) neprilysin (SEQ ID NO:4)

MCRSESQMDITDINTPKPKKKQRWTPLEISLSVLVLLLTVIAVTMIALYA
TYDDGICKSSDCIKSAARLIQNMDATAEPCTDEFKYACGGWLKRNVIPET
SSRYSNFDILRDELEVILKDVLQEPKTEDIVAVQKAKTLYRSCVNETAID
SRGGQPLLKLLPDVYGWPVATQNWEQTYGTSWSAEKSIAQLNSNYGKKVL
INFFVGTDDKNSMNHIIHIDQPRLGLPSRDYYECTGIYKEACTAYVDFMI
AVAKLIRQEEGLPIDENQISVEMNKVMELEKEIANATTKSEDRNDPMLLY
NKMTLAQIQNNFSLEINGKPFSWSNFTNEIMSTVNINIPNEEDVVVYAPE
YLIKLKPILTKYFPRDFQNLFSWRFIMDLVSSLSRTYKDSRNAFRKALYG
TTSESATWRRCANYVNGNMENAVGRLYVEAAFAGESKHVVEDLIAQIREV
FIQTLDDLTWMDAETKKKAEEKALAIKERIGYPDDIVSNDNKLNNEYLEL
NYKEDEYFENIIQNLKFSQSKQLKKLREKVDKDEWITGAAIVNAFYSSGR
NQIVFPAGILQPPFFSAQQSNSLNYGGIGMVTGHEITHGFDDNGRNFNKD
GDLVDWWTQQSANNFKEQSQCMVYQYGNFSWDLAGGQHLNGINTLGENIA
DNGGIGQAYRAYQNYVKKNGEEKLLPGIDLNHKQLFFLNFAQVWCGTYRP
EYAVNSIKTDVHSPGNFRIIGSLQNSVEFSEAFQCPKNSYMNPEKKCRVW

From GenBank Accession Number AAC28366 Yellow Perch (Perca flavescens) neprilysin (SEQ ID NO:5)

MPIYIIDRKFPDTSGELIQPAAEAGDLRMMETNPPKSAKKPRWTSLEVGL
TTIVSLLFIVIVALIILFATQKTDEICTTGDCTQSASRLIENMDDSVDPC
DNFYQYACGGWLKKNIIPETSSRYSTFDILRDELEVILKGVLEKTDEGEA
TLSTRAKTLYKSCTNESLIELRGGAPLLDMLPDVFEWPVAVDNWETNYGK
TWRLEDVIAKLNEKYGTQLLVNFFVGTDDRDSNSYIIHFDQQTNLGLLSR
DYYACTGPYAEACRAYEKFMIDLAKLIRIDRGLNISETDIREEVKRVMDL
ERDIANATDTPEDRNNPVLLYNKMELGDLNANFTLEVESQVFDWSYFTAK
IMDTVNISVPDTEKVINYSPNYYRRLNLILARYNKRDLQNYMVWRFAMNM
VVGLSRSYRDTRKAFRKALSGTTSEAAVWRQCALYVNNNMDNAVGRLYVQ
EAFSEKSKELMEEMIKDIREVFISNLDDLTWMDAETKKAAEEKARAIRER
IGYSDNIKDDKYLNNEYNDLAYSAEEYFENILQNLEYVQKKRLRKLRVKV
NKEEWVTGAAVVNAFYSSSKNQIVFPAGILQPPFFSKGQAKSLNYGGIGM
VIGHEITHGFDDNGRNYDKDGDLKDWWTPGSTDRFLDLSKCIVNQYGNFS
WDLANGLHLNGNNTLGENIADNGGIRQAYQAYKNYVEKHGEEPSLPGINL
SHNQLFFLNFAQVWCGTHRPEQAVNSIKVDVHSPGKFRVLGSLQNFPEFA
KAFNCKKNSYMVPANICRVW

From GenBank Accession Number NP_{—}001004412 Chicken (Gallus gallus) neprilysin (SEQ ID NO:6)

MGKSESQMDITEMNAPKPKKKLRWSGLEIGLTVVVILLAIVAITMIVLYA
TYDDGVCKTSDCIKSAARIIENMDTTAEPCNDFYQYACGGWLKRNVIPET
SSRYSNFDILRDELEVVLKDVLDTPSSNDITAVQKAKTLYRSCINETTID
SRGGMPLISLLANLSEWPVATNNWESSYGAAWTAETAIAQLTSRYGKKVL
INFFVGTDDKNSTAHIIHIDQPGLGLPSRDYYECTGAYQEACSAYVDFMI
SVAKLILQERNITFNETQIAEEMKRVMDLEKEIANATTKSEDRNDPLLLY
NKMTLAQLQNNFSLEINHMAFNWSKFINNIMSTVQIDVENTEHVVVYDPE
YLTKLKSILNKYTPRELQNYMIWRFVMDLVNSLSRNYKDTRNAFRKALYG
TTSETAVWRRCANYVNGNMENAVGRLYVQEAFAGDSKHVVEEMIADIRGV
FIETLDDLTWMDAETKKKAEQKATAIKERIGYPDEIMTDDSKLNSEYQEL
NYKEEEYFENIIQNLVFTQKKRLKKLREKVDKEEWISGAAVVNAFYSASR
NQIVFPAGILQPPFFSASQPKSLNYGGIGMVIGHEITHGFDDNGRNFNEN
GDLVDWWTEESARNFKDLSQCMVYQYGNFSWDLAGGQQLSGINTLGENIA
DNGGVRQAYKAYENFVKKNGKEKLLPGLDMNHQQLFFLNFAQVWCGTYRP
EYAVNSIKTDVHSPGKFRVIGSLQNSPEFSEAFSCTTKSYMDPAKKCRVW

From GenBank Accession Number JC7265 Rat (Rattus norvegicus) neprilysin II (SEQ ID NO:7)

MCKSESSVGMMERADNCGRRRLGFVECGLLVLLTLLLMGAIVTLGVFYSI
GKQLPLLNSLLHVSRHERTVVKRVLRDSSQKSDICTTPSCVIAAARILQN
MDQSKKPCDNFYQYACGGWLRHHVIPETNSRYSVFDILRDELEVILKGVL
EDSSVQHRPAVEKAKTLYRSCMNQSVIEKRDSEPLLNVLDMIGGWPVAMD
KWNETMGPKWELERQLAVLNSQFNRRVLIDLFIWNDDQNSSRHVIYIDQP
TLGMPSREYYFKEDSHRVREAYLQFMTSVATMLRRDLNLPGETDLVQEEM
AQVLHLETHLANATVPQEKRHDVTALYHRMGLEELQERFGLKGFNWTLFI
QNVLSSVQVELLPNEEVVVYGIPYLENLEEIIDVFPAQTLQNYLVWRLVL
DRIGSLSQRFKEARVDYRKALYGTTMEEVRWRECVSYVNSNMESAVGSLY
IKRAFSKDSKSIVSELIEKIRSVFVDNLDELNWMDEESKKKAQEKALNIR
EQIGYPDYILEDNNRHLDEEYSSLTFSEDLYFENGLQNLKNNAQRSLKKL
REKVDQNLWIIGAAVVNAFYSPNRNLIVFPAGILQPPFFSKDQPQALNFG
GIGMVIGHEITHGFDDNGRNFDKNGNMLDWWSNFSARHFRQQSQCMIYQY
SNFSWELADNQNVNGFSTLGENIADNGGVRQAYKAYLQWLAEGGRDQRLP
GLNLTYAQLFFINYAQVWCGSYRPEFAIQSIKTDVHSPLKYRVLGSLQNL
PGFSEAFHCPRGSPMHPMNRCRIW

From GenBank Accession Number AAL08942 Human (Homo sapiens) neprilysin II (SEQ ID NO:8)

MVESAGRAGQKRPGFLEGGLLLLLLLVTAALVALGVLYADRRGKQLPRLA
SRLCFLQEERTFVKRKPRGIPEAQEVSEVCTTPGCVIAAARILQNMDPTT
EPCDDFYQFACGGWLRRHVIPETNSRYSIFDVLRDELEVILKAVLENSTA
KDRPAVEKARTLYRSCMNQSVIEKRGSQPLLDILEVVGGWPVAMDRWNET
VGLEWELERQLALMNSQFNRRVLIDLFIWNDDQNSSRHIIYIDQPTLGMP
SREYYFNGGSNRKVREAYLQFMVSVATLLREDANLPRDSCLVQEDMVQVL
ELETQLAKATVPQEERHDVIALYHRMGLEELQSQFGLKGFNWTLFIQTVL
SSVKIKLLPDEEVVVYGIPYLQNLENIIDTYSARTIQNYLVWRLVLDRIG
SLSQRFKDTRVNYRKALFGTMVEEVRWRECVGYVNSNMENAVGSLYVREA
FPGDSKSMVRELIDKVRTVFVETLDELGWMDEESKKKAQEKAMSIREQIG
HPDYILEEMNRRLDEEYSNLNFSEDLYFENSLQNLKVGAQRSLRKLREKV
DPNLWIIGAAVVNAFYSPNRNQIVFPAGILQPPFFSKEQPQALNFGGIGM
VIGHEITHGFDDNGRNFDKNGNMMDWWSNFSTQHFREQSECMIYQYGNYS
WDLADEQNVNGFNTLGENIADNGGVRQAYKAYLKWMAEGGKDQQLPGLDL
THEQLFFINYAQVWCGSYRPEFAIQSIKTDVHSPLKYRVLGSLQNLAAFA
DTFHCARGTPMHPKERCRVW

From GenBank Accession Number XM_{—}534313 Dog (Canis familiaris) neprilysin (SEQ ID NO:9)

MGRSESQMDITDISTPRPKKQRWTSLEISLSVLVLLLTIIAVTMIALYAT
YDAARLIQNMDATAEPCTDFFKYACGGWLKRNVIPETSSRYSNFDILRDE
LEVVLKDVLQEPKTEDIVAVQKAKTLYRSCINESAIDSRGGQPLLSLLPD
IYDWPVATDNWEQTYGTSWTAEKSIAQLNSKYGKKVILNFFVGTDDKNSV
NHIIHIDQPRLGLPSRDYYVCTGIYEEGTTEVETLGSGKPLPPTRVSGPR
GIFRPGLRDVLQGCVVGALAPSSQWKGVGMWDDISHPGNGLIRKEKGLLI
DENQLSLEMNRVMELEKEIASATTKPEDRNDPMLLYNKMTLAQIQNNFTL
EIDGKPFSWSNFTNEIMSTVNINIPNEEEVVVYAPEYLTKLKLILTKYSS
RDLQNLMSWRFIMDLVSSLSRNYKESRNAFRKALYGTTSETATWRRCANY
VNGNMENAVGRLYVEAAFAGESKHVVEDLITQIRAVFIQTLDDLTWMDAE
TKKKAEEKALAIKERIGYPDDIISNDSKLDNEYLELNYREDEYEENIIQN
LKFSQNKQLKKLREKVDKDEWISGAAVVNAFYSSGRNQIVFPAGILQPPF
FSALQSNSLNYGGIGMVIGHEITHGFDDNGRNFNKDGDLVDWWTQQSANN
FKDQSQCMVYQYGNFSWDLAGGQHLNGINTLGENIADNGGIGQAYRAYQN
YVKKNGEEKLLPGLDLNHKQLFFLNFAQVWCGTYRPEYAVNSIKTDVHSP
GNFRIIGTLQNSPEFSEAFHCRKNSYMNPEKKCRVW

From GenBank Accession Number AF336981 Human (Homo sapiens) neprilysin-like peptide (SEQ ID NO:10)

MVESAGRAGQKRPGFLEGGLLLLLLLVTAALVALGVLYADRRGKQLPRLA
SRLCFLQEERTFVKRKPRGIPEAQEVSEVCTTPGCVIAAARILQNMDPTT
EPCDDFYQFACGGWLRRHVIPETNSRYSIFDVLRDELEVILKAVLENSTA
KDRPAVEKARTLYRSCMNQSVIEKRGSQPLLDILEVVGGWPVAMDRWNET
VGLEWELERQLALMNSQFNRRVLIDLFIWNDDQNSSRHIIYIDQPTLGMP
SREYYFNGGSNRKVREAYLQFMVSVATLLREDANLPRDSCLVQEDMVQVL
ELETQLAKATVPQEERHDVIALYHRMGLEELQSQFGLKGFNWTLFIQTVL
SSVKIKLLPDEEVVVYGIPYLQNLENIIDTYSARTIQNYLVWRLVLDRIG
SLSQRFKDTRVNYRKALFGTMVEEVRWRECVGYVNSNMENAVGSLYVREA
FPGDSKSMVRELIDKVRTVFVETLDELGWMDEESKKKAQEKAMSIREQIG
HPDYILEEMNRRLDEEYSNLNFSEDLYFENSLQNLKVGAQRSLRKLREKV
DPNLWIIGAAVVNAFYSPNRNQIVFPAGILQPPFFSKEQPQALNFGGIGM
VIGHEITHGFDDNGRNFDKNGNMMDWWSNFSTQHFREQSECMIYQYGNYS
WDLADEQNVNGFNTLGENIADNGGVRQAYKAYLKWMAEGGKDQQLPGLDL
THEQLFFINYAQVWCGSYRPEFAIQSIKTDVHSPLKYRVLGSLQNLAAFA
DTFHCARGTPMHPKERCRVW

### 7.0 REFERENCES

The following references, to the extent that they provide exemplary procedural or other details supplementary to those set forth herein, are specifically incorporated herein by reference.
U.S. Pat. No. 4,237,224, issued Dec. 2, 1980.
U.S. Pat. No. 4,554,101, issued Nov. 19, 1985.
U.S. Pat. No. 4,683,195, issued Jul. 28, 1987.
U.S. Pat. No. 4,683,202, issued Jul. 28, 1987.
U.S. Pat. No. 4,800,159, issued Jan. 24, 1989.
U.S. Pat. No. 4,883,750, issued Nov. 28, 1989.
U.S. Pat. No. 4,987,071, issued Jan. 22, 1991.
U.S. Pat. No. 5,145,684, issued Sep. 8, 1992.
U.S. Pat. No. 5,334,711, issued Aug. 2, 1994.
U.S. Pat. No. 5,354,855, issued Oct. 11, 1994.
U.S. Pat. No. 5,399,363, issued Mar. 21, 1995.
U.S. Pat. No. 5,466,468, issued Nov. 14, 1995.
U.S. Pat. No. 5,543,158, issued Apr. 6, 1996.
U.S. Pat. No. 5,552,157, issued Sep. 3, 1996.
U.S. Pat. No. 5,565,213, issued Oct. 15, 1996.
U.S. Pat. No. 5,567,434, issued Oct. 22, 1996.
U.S. Pat. No. 5,602,306, issued Feb. 11, 1997.
U.S. Pat. No. 5,631,359, issued May 20, 1997.
U.S. Pat. No. 5,639,940, issued Jun. 17, 1997.
U.S. Pat. No. 5,641,515, issued Jun. 24, 1997.
U.S. Pat. No. 5,656,016, issued Aug. 12, 1997.
U.S. Pat. No. 5,697,899, issued Dec. 16, 1997.
U.S. Pat. No. 5,720,936, issued Feb. 24, 1998.
U.S. Pat. No. 5,738,868, issued Apr. 14, 1998.
U.S. Pat. No. 5,741,516, issued Apr. 21, 1998.
U.S. Pat. No. 5,770,219, issued Jun. 23, 1998.
U.S. Pat. No. 5,779,708, issued Jul. 14, 1998.
U.S. Pat. No. 5,783,208, issued Jul. 21, 1998.
U.S. Pat. No. 5,789,655, issued Aug. 4, 1998.
U.S. Pat. No. 5,795,587, issued Aug. 18, 1998.
U.S. Pat. No. 5,797,898, issued Aug. 25, 1998.
Int. Pat. Appl. No. PCT/US87/00880.
Int. Pat. Appl. No. PCT/US88/10315.
Int. Pat. Appl. No. PCT/US89/01025.
Int. Pat. Appl. Publ. No. WO 89/06700.
Int. Pat. Appl. Publ. No. WO 91/03162.
Int. Pat. Appl. Publ. No. WO 92/07065.
Int. Pat. Appl. Publ. No. WO 93/15187.
Int. Pat. Appl. Publ. No. WO 93/23569.
Int. Pat. Appl. Publ. No. WO 94/02595.
Int. Pat. Appl. Publ. No. WO 94/13688.
Eur. Pat. Appl. Publ. No. EP 0329822.
Eur. Pat. Appl. Publ. No. EP 0360257.
Eur. Pat. Appl. Publ. No. EP 92110298.4.
Eur. Pat. Appl. Publ. No. 320,308.
Great Britian Appl. No. 2202328.
Allen and Choun, “Large unilamellar liposomes with low uptake into the reticuloendothelial system,” FEBS Lett., 223:42-46, 1987.
Angel, Bauman, Stein, Dellus, Rahmsdorf and Herrlich, “12-O-tetradecanoyl-phorbol-13-acetate induction of the human collagenase gene is mediated by an inducible enhancer element located in the 5′ flanking region,” Mol. Cell. Biol., 7:2256, 1987a.
Angel, Imagawa, Chiu, Stein, Imbra, Rahmsdorf, Jonat, Herrlich and Karin, “Phorbol ester-inducible genes contain a common cis element recognized by a TPA-modulated trans-acting factor,” Cell, 49:729, 1987b.
Atchinson et al., Science, 194:754-756, 1965.
Atchison and Perry, “Tandem kappa immunoglobulin promoters are equally active in the presence of the kappa enhancer: Implications for model of enhancer function,” Cell, 46:253, 1986.
Atchison and Perry, “The role of the kappa enhancer and its binding factor NF-kappa B in the developmental regulation of kappa gene transcription,” Cell, 48:121, 1987.
Balazsovits et al., “Analysis of the effect of liposome encapsulation on the vesicant properties, acute and cardiac toxicities, and antitumor efficacy of doxorubicin,” Cancer Chemother. Pharmacol., 23:81-86, 1989.
Banerji, Olson and Schaffner, “A lymphocyte-specific cellular enhancer is located downstream of the joining region in immunoglobulin heavy-chain genes,” Cell, 35:729, 1983.
Banerji, Rusconi and Schaffner, “Expression of a β-globin gene is enhanced by remote SV40 DNA sequences,” Cell, 27:299, 1981.
Bartlett and Samulski, “Fluorescent viral vectors: a new technique for the pharmacological analysis of gene therapy,” Nat. Med., 4:635-7, 1998.
Berkhout, Silverman and Jeang, “tat trans-activates the human immunodeficiency virus through a nascent RNA target,” Cell, 59:273, 1989.
Berns et al., In: Virus Persistence, Mehay et al. (Ed.), Cambridge Univ. Press, pp. 249-265, 1982.
Berns, Pinkerton, Thomas, Hoggan, “Detection of adeno-associated virus (AAV)-specific nucleotide sequences in DNA isolated from latently infected Detroit 6 cells,” Virol., 68:556-560, 1975.
Bjorbaek et al., Endocrinol., 140:2035-2043, 1999.
Bjorbaek et al., Mol. Cell., 1:619-625, 1998.
Blanar, Baldwin, Flavell and Sharp, “A gamma-interferon-induced factor that binds the interferon response sequence of the MHC Class I gene, H-2 Kb,” EMBO J., 8:1139, 1989.
Bodine and Ley, “An enhancer element lies 3′ to the Human A gamma globin gene,” EMBO J, 6:2997, 1987.
Boshart, Weber, Jahn, Dorsch-Hasler, Fleckenstein and Schaffner, “A very strong enhancer is located upstream of an immediate early gene of human cytomegalovirus,” Cell, 41:521, 1985.
Bosze, Thiesen and Charnay, “A transcriptional enhancer with specificity for erythroid cells is located in the long terminal repeat of the friend murine leukemia virus,” EMBO J, 5:1615, 1986.
Bourlais, Acar, Zia, Sado, Needham, Leverge, “Ophthalmic drug delivery systems—recent advances,” Prog. Retin Eye Res., 17:33-58, 1998.
Braddock, Chambers, Wilson, Esnouf, Adams, Kingsman and Kingsman, “HIV-I tat activates presynthesized RNA in the nucleus,” Cell, 58:269, 1989.
Bulla and Siddiqui, “The Hepatitis B virus enhancer modulates transcription of the Hepatitis B virus surface-antigen gene from an internal location,” J. Virol., 62:1437, 1986.
Buller, Janik, Sebring, Rose, “Herpes simplex virus types 1 and 2 completely help adenovirus-associated virus replication,” J. Virol., 40:241-247, 1981.
Caldovic and Hackett Jr., “Development of position-independent expression vectors and their transfer into transgenic fish,” Mol. Mar. Biol. Biotechnol., 4:51-61, 1995.
Campbell and Villarreal, “Functional analysis of the individual enhancer core sequences of polyoma virus: cell-specific uncoupling of DNA replication from transcription,” Mol. Cell. Biol., 8:1993, 1988.
Campere and Tilghman, “Postnatal repression of the α-fetoprotein gene is enhancer independent,” Genes and Dev., 3:537, 1989.
Campo, Spandidos, Lang and Wilkie, “Transcriptional control signals in the genome of bovine papilloma virus Type 1,” Nature, 303:77, 1983.
Capecchi, “High efficiency transformation by direct microinjection of DNA into cultured mammalian cells,” Cell, 22:479-488, 1980.
Carter et al., In: The Parvoviruses, K. I. Berns (Ed.), Plenum, N.Y., pp. 153-207, 1983.
Carver, Dalrymple, Wright, Cottom, Reeves, Gibson, Keenan, Barrass, Scott, Colman, et al., “Transgenic livestock as bioreactors: stable expression of human α-1-antitrypsin by a flock of sheep,” Biotechnology N.Y., 11: 1263-1270, 1993.
Cech et al., “In vitro splicing of the ribosomal RNA precursor of Tetrahymena: involvement of a guanosine nucleotide in the excision of the intervening sequence,” Cell, 27:487-496, 1981.
Celander and Haseltine, “Glucocorticoid regulation of murine leukemia virus transcription elements is specified by determinants within the viral enhancer region,” J Virology, 61:269, 1987.
Celander, Hsu and Haseltine, “Regulatory elements within the murine leukemia virus enhancer regions mediate glucocorticoid responsiveness,” J Virology, 62:1314, 1988.
Chandler, Maler and Yamamoto, “DNA sequences bound specifically by glucocorticoid receptor in vitro render a heterlogous promoter hormone responsive in vivo,” Cell, 33:489, 1983.
Chandran, Roy, Mishra, “Recent trends in drug delivery systems: liposomal drug delivery system—preparation and characterisation,” Indian J. Exp. Biol., 35:801-809, 1997.
Chang, Erwin and Lee, “Glucose-regulated protein (GRP94 and GRP78) genes share common regulatory domains and are coordinately regulated by common trans-acting factors,” Mol. Cell. Biol., 9:2153, 1989.
Chatterjee, Lee, Rentoumis and Jameson, “Negative regulation of the thyroid-stimulating hormone α gene by thyroid hormone: Receptor interaction adjacent to the TATA box,” Proc. Natl. Acad. Sci. U.S.A., 86:9114, 1989.
Chen and Okayama, “High-efficiency transformation of mammalian cells by plasmid DNA,” Mol. Cell. Biol., 7:2745-2752, 1987.
Chen et al., “Multitarget-ribozyme directed to cleave at up to nine highly conserved HIV-1 env RNA regions inhibits HIV-1 replication—potential effectiveness against most presently sequenced HIV-1 isolates,” Nucl. Acids Res., 20:4581-89, 1992.
Cheung, Hoggan, Hauswirth, Bems, “Integration of the adeno-associated virus genome into cellular DNA in latently infected human Detroit 6 cells,” J. Virol., 33:739-48, 1980.
Choi, Chen, Kriegler and Roninson, “An altered pattern of cross-resistance in multi-drug-resistant human cells results from spontaneous mutations in the MDR-1 (P-glycoprotein) gene,” Cell, 53:519, 1988.
Chowrira and Burke, “Extensive phosphorothioate substitution yields highly active and nuclease-resistant hairpin ribozymes,” Nuc. Acids Res., 20:2835-40, 1992.
Clark et al., Hum. Gene Ther., 6:1329-41, 1995.
Cohen, Walter and Levinson, “A repetitive sequence element 3′ of the human c-Ha-ras1 gene has enhancer activity,” J. Cell. Physiol., 5:75, 1987.
Collins and Olive, “Reaction conditions and kinetics of self-cleavage of a ribozyme derived from Neurospora VS RNA,” Biochem., 32:2795-99, 1993.
Costa, Lai, Grayson and Darnell, “The cell-specific enhancer of the mouse transthyretin (prealbumin) gene binds a common factor at one site and a liver-specific factor(s) at two other sites,” Mol. Cell. Biol., 8:81, 1988.
Coune, “Liposomes as drug delivery system in the treatment of infectious diseases: potential applications and clinical experience,” Infection, 16:141-47, 1988.
Couvreur et al., “Nanocapsules, a new lysosomotropic carrier,” FEBS Lett., 84:323-26, 1977.
Couvreur et al., “Tissue distribution of antitumor drugs associated with polyalkylcyanoacrylate nanoparticles,” J. Pharm. Sci., 69:199-202, 1980.
Couvreur, “Polyalkyleyanoacrylates as colloidal drug carriers,” Crit. Rev. Ther. Drug Carrier Syst., 5:1-20, 1988.
Cozzi, Tucker, Langford, Pino-Chavez, Wright, O'Connell, Young, Lancaster, McLanghlin, Hunt, Bordin, White, “Characterization of pigs transgenic for human decay-accelerating factor,” Transplantation, 641383-92, 1997.
Cripe, Haugen, Turk, Tabatabai, Schmid, Durst, Gissmann, Roman and Turek, “Transcriptional regulation of the human papilloma virus-16 E6-E7 promoter by a keratinocyte-dependent enhancer, and by viral E2 trans-activator and repressor gene products: Implications for cervical carcinogenesis,” EMBO J., 6:3745, 1987.
Cukor et al., In: The Paroviruses, K. I. Berns (Ed.), Plenum, N.Y., pp. 33-66, 1984.
Culotta and Hamer, “Fine mapping of a mouse metallothionein gene metal-response element,” Mol. Cell. Biol., 9:1376, 1989.
Curiel, Agarwal, Wagner and Cotten, “Adenovirus enhancement of transferrin-polylysine-mediated gene delivery,” Proc. Natl. Acad. Sci. USA, 88:8850-54, 1991.
Dandolo, Blangy and Kamen, “Regulation of polyma virus transcription in murine embryonal carcinoma cells,” J Virology, 47:55, 1983.
De Villiers, Schaffner, Tyndall, Lupton and Kamen, “Polyoma virus DNA replication requires an enhancer,” Nature, 312:242, 1984.
DeRisi, Iyer and Brown, “Exploring the metabolic and genetic control of gene expression on a genomic scale,” Science, 278:680-86, 1997.
Deschamps, Meijlink and Verma, “Identification of a transcriptional enhancer element upstream from the proto-oncogene Fos,” Science, 230:1174, 1985.
Douglas, Davis and Illum, “Nanoparticles in drug delivery,” Crit. Rev. Ther. Drug Carrier Syst., 3:233-61, 1987.
Dropulic, Lin, Martin and Jeang, “Functional characterization of a U5 ribozyme: intracellular suppression of human immunodeficiency virus type 1 expression,” J. Virol., 66:1432-41, 1992.
Ebert, Selgrath, DiTullio, Denman, Smith, Memon, Schindler, Monastersky, Vitale and Gordon, “Transgenic production of a variant of human tissue-type plasminogen activator in goat milk: generation of transgenic goats and analysis of expression,” Biotechnology N.Y., 9:835-38, 1991.
Edbrooke, Burt, Cheshire and Woo, “Identification of cis-acting sequences responsible for phorbol ester induction of human serum amyloid a gene expression via a nuclear-factor-κB-like transcription factor,” Mol. Cell. Biol., 9:1908, 1989.
Edlund, Walker, Barr and Rutter, “Cell-specific expression of the rat insulin gene: Evidence for role of two distinct 5′ flanking elements,” Science, 230:912, 1985.
Elroy-Stein and Moss, “Cytoplasmic expression system based on constitutive synthesis of bacteriophage T7 RNA polymerase in mammalian cells,” Proc. Natl. Acad. Sci. USA, 87:6743-47, 1990.
Emilsson et al., FEBS Letters, 455:170-74, 1999.
Faller and Baltimore, “Liposome encapsulation of retrovirus allows efficient super infection of resistant cell lines,” J. Virol., 49:269-72, 1984.
Fallon, “Transgenic insect cells: mosquito cell mutants and the dihydrofolate reductase gene,” Cytotechnology, 20:23-31, 1996.
Fechheimer et al., “Transfection of mammalian cells with plasmid DNA by scrape loading and sonication loading,” Proc. Natl. Acad. Sci. USA, 84:8463-67, 1987.
Feng and Holland, “HIV-I tat trans-activation requires the loop sequence within tar,” Nature, 334:6178, 1988.
Firak and Subramanian, “Minimal transcription enhancer of simian virus 40 is a 74-base-pair sequence that has interacting domains,” Mol. Cell. Biol., 6:3667, 1986.
Flotte et al., “Stable in vivo expression of the cystic fibrosis transmembrane conductance regulator with an adeno-associated virus vector,” Proc. Natl. Acad. Sci. USA, 90:10613-17, 1993.
Foecking and Hofstetter, “Powerful and versatile enhancer-promoter unit for mammalian expression vectors,” Gene, 45:101-05, 1986.
Forster and Symons, “Self-cleavage of plus and minus RNAs of a virusoid and a structural model for the active sites,” Cell, 49:211-20, 1987.
Fraley et al., “Entrapment of a bacterial plasmid in phospholipid vesicles: Potential for gene transfer,” Proc. Natl. Acad. Sci. USA, 76:3348-52, 1979.
Franz, Mueller, Haartong, Frey and Katus, “Transgenic animal models: new avenues in cardiovascular physiology,” J. Mol. Med., 75:115-19, 1997.
Frederich et al., Nat. Med., 1:1311-14, 1995.
Fresta and Puglisi, “Application of liposomes as potential cutaneous drug delivery systems. In vitro and in vivo investigation with radioactively labelled vesicles,” J Drug Target, 4:95-101, 1996.
Frohman, Downs, Kashio and Brinster, “Tissue distribution and molecular heterogeneity of human growth hormone-releasing factor in the transgenic mouse,” Endocrinology, 127:2149-56, 1990.
Fromm, Taylor and Walbot, “Expression of genes transferred into monocot and dicot plant cells by electroporation,” Proc. Natl. Acad. Sci. USA, 82:5824-28, 1985.
Fujita, Shibuya, Hotta, Yamanishi and Taniguchi, “Interferon-P gene regulation: tandemly repeated sequences of a synthetic 6-bp oligomer function as a virus-inducible enhancer,” Cell, 49:357, 1987.
Gabizon and Papahadjopoulos, “Liposomes formulations with prolonged circulation time in blood and enhanced uptake by tumors,” Proc. Natl. Acad. Sci. USA, 85:6949-53, 1988.
Gao and Huang, “Cytoplasmic expression of a reporter gene by co-delivery of T7 RNA polymerase and T7 promoter sequence with cationic liposomes,” Nuc. Acids Res., 21:2867-72, 1993.
Gerlach et al., “Construction of a plant disease resistance gene from the satellite RNA of tobacco rinspot virus,” Nature (London), 328:802-05, 1987.
Gilles, Morris, Oi and Tonegawa, “A tissue-specific transcription enhancer element is lcoated in the major intron of a rearranged immunoglobulin heavy-chain gene,” Cell, 33:717, 1983.
Gloss, Bernard, Seedorf and Klock, “The upstream regulatory region of the human papilloma virus-16 contains an E2 protein-independent enhancer which is specific for cervical carcinoma cells and regulated by glucocorticoid hormones,” EMBO J, 6:3735, 1987.
Godbout, Ingram and Tilghman, “Fine-structure mapping of the three mouse a-fetoprotein gene enhancers,” Mol. Cell. Biol., 8:1169, 1988.
Goodboum and Maniatis, “Overlapping positive and negative regulatory domains of the human β-interferon gene,” Proc. Natl. Acad. Sci. USA, 85:1447, 1988.
Goodbourn, Burstein and Maniatis, “The human α-interferon gene enhancer is under negative control,” Cell, 45:601, 1986.
Gopal, “Gene transfer method for transient gene expression, stable transfection, and cotransfection of suspension cell cultures,” Mol. Cell Biol., 5:1188-90, 1985.
Graham and van der Eb, “Transformation of rat cells by DNA of human adenovirus 5,” Virol., 54:536-39, 1973.
Greene, Bohnlein and Ballard, “HIV-1, and normal T-cell growth: Transcriptional strategies and surprises,” Immunol. Today, 10:272, 1989.
Grimm et al., Hum. Gene Ther., 9:2745-60, 1998.
Grosschedl and Baltimore, “Cell-type specificity of immunoglobulin gene expression is regulated by at least three DNA sequence elements,” Cell, 41:885, 1985.
Guerrier-Takada, Gardiner, Marsh, Pace and Altman, “The RNA moiety of ribonuclease P is the catalytic subunit of the enzyme,” Cell, 35:849, 1983.
Haberman et al., Gene Ther., 5:1604-11, 1998.
Hampel and Tritz, “RNA catalytic properties of the minimum (−)s TRSV sequence,” Biochem., 28:4929, 1989.
Hampel, Tritz, Hicks and Cruz, “‘Hairpin’ catalytic RNA model: evidence for helices and sequence requirement for substrate RNA,” Nucl. Acids Res., 18:299, 1990.
Handa, Shiroki and Shimojo, “Establishment and characterization of KB cell lines latently infected with adeno-associated virus type 1,” Virol., 82:84-92, 1977.
Harland and Weintraub, “Translation of mRNA injected into Xenopus oocytes is specifically inhibited by antisense RNA,” J. Cell Biol., 101:1094-99, 1985.
Haskell and Bowen, “Efficient production of transgenic cattle by retroviral infection of early embryos,” Mol. Reprod. Dev., 40:386-90, 1995.
Haslinger and Karin, “Upstream promoter element of the human metallothionein-II gene can act like an enhancer element,” Proc. Natl. Acad. Sci. USA, 82:8572, 1985.
Hauber and Cullen, “Mutational analysis of the trans-activiation-responsive region of the human immunodeficiency virus Type I long terminal repeat,” J. Virol., 62:673, 1988.
Heath and Martin, “The development and application of protein-liposome conjugation techniques,” Chem. Phys. Lipids, 40:347-58, 1986.
Heath et al., “Liposome-mediated delivery of pteridine antifolates to cells: in vitro potency of methotrexate and its α and gamma substituents,” Biochim. Biophys. Acta, 862:72-80, 1986.
Hen, Borrelli, Fromental, Sassone-Corsi and Chambon, “A mutated polyoma virus enhancer which is active in undifferentiated embryonal carcinoma cells is not repressed by adenovirus-2 E1A products,” Nature, 321:249, 1986.
Henry-Michelland et al., “Attachment of antibiotics to nanoparticles; Preparation, drug-release and antimicrobial activity in vitro,” Int. J. Pharm., 35:121-27, 1987.
Hensel, Meichle, Pfizenmaier and Kronke, “PMA-responsive 5′ flanking sequences of the human TNF gene,” Lymphokine Res., 8:347, 1989.
Hermonat, Labow, Wright, Bems and Muzyczka, “Genetics of adeno-associated virus: isolation and preliminary characterization of adeno-associated virus type 2 mutants,” J. Virol., 51:329-39, 1984.
Herr and Clarke, “The SV40 enhancer is composed of multiple functional elements that can compensate for one another,” Cell, 45:461, 1986.
Hirochika, Browker and Chow, “Enhancers and trans-acting E2 transcriptional factors of papilloma viruses,” J. Virol., 61:2599, 1987.
Hirsch, Gaugler, Deagostini-Bauzin, Bally-Cuif and Gordis, “Identification of positive and negative regulatory elements governing cell-type-specific expression of the neural-cell-adhesion-molecule gene,” Mol. Cell. Biol., 10:1959, 1990.
Hoggan et al., In: Proceeding of the Fourth Lepetit Colloquium, Cacoyac, Mexico, North Holland, Amsterdam, pp. 243-249, 1972.
Hoggan, Fed. Proc., 24:248, 1965.
Holbrook, Gulino and Ruscetti, “cis-acting transcriptional regulatory sequences in the Gibbon ape leukemia virus (GALV) long terminal repeat,” Virology, 157:211, 1987.
Hoover et al., Eds., In: Remington 's Pharmaceutical Sciences, 16^{th} Edition, Mack Publishing Co., Easton, Pa., 1980.
Horlick and Benfield, “The upstream muscle-specific enhancer of the rat muscle creatine kinase gene is composed of multiple elements,” Mol. Cell. Biol., 9:2396, 1989.
Huang, Ostrowski, Berard and Hagar, “Glucocorticoid regulation of the Ha-MuSV p21 gene conferred by sequences from mouse mammary tumor virus,” Cell, 27:245, 1981.
Hwang, Lim and Chae, “Characterization of the s-phase-specific transcription regulatory elements in a DNA-replication-independent testis-specific H₂B (TH2B) histone gene,” Mol. Cell. Biol., 10:585, 1990.
Imagawa, Chiu and Karin, “Transcription factor AP-2 mediates induction by two different signal-transduction pathways: Protein kinase C and cAMP,” Cell, 51:251, 1987.
Imaizumi et al., “Liposome-entrapped superoxide dismutase reduces cerebral infarction in cerebral ischemia in rats,” Stroke, 21:1312-17, 1990a.
Imaizumi et al., “Liposome-entrapped superoxide dismutase ameliorates infarct volume in focal cerebral ischemia,” Acta. Neurochirurgica Suppl., 51:236-38, 1990b.
Imbra and Karin, “Phorbol ester induces the transcriptional stimulatory activity of the SV40 enhancer,” Nature, 323:555, 1986.
Imler, Lemaire, Wasvlyk and Waslyk, “Negative regulation contributes to tissue specificity of the immunoglobulin heavy-chain enhancer,” Mol. Cell. Biol., 7:2558, 1987.
Imperiale and Nevins, “Adenovirus 5 E2 transcription unit: An E1A-inducible promoter with an essential element that functions independently of position or orientation,” Mol. Cell. Biol., 4:875, 1984.
Jaeger, Turner and Zuker, “Improved predictions of secondary structures for RNA,” Proc. Natl. Acad. Sci. USA, 86:7706-10, 1989.
Jakobovits, Smith, Jakobovits and Capon, “A discrete element 3′ of human immunodeficiency virus 1 (HIV-1) and HIV-2 mRNA initiation sites mediates transcriptional activation by an HIV trans-activator,” Mol. Cell. Biol., 8:2555, 1988.
Jameel and Siddiqui, “The human Hepatitis B virus enhancer requires transacting cellular factor(s) for activity,” Mol. Cell. Biol., 6:710, 1986.
Jaynes, Johnson, Buskin, Gartside and Hauschka, “The muscle creatine kinase gene is regulated by multiple upstream elements, including a muscle-specific enhancer,” Mol. Cell. Biol., 8:62, 1988.
Johnson, Wold and Hauschka, “Muscle creatine kinase sequence elements regulating skeletal and cardiac muscle expression in transgenic mice,” Mol. Cell. Biol., 9:3393, 1989a.
Joyce, “RNA evolution and the origins of life,” Nature, 338:217-44, 1989.
Kadesch and Berg, “Effects of the position of the simian virus 40 enhancer on expression of multiple transcription units in a single plasmid,” Mol. Cell. Biol., 6:2593, 1986.
Kang, et al., “Up-regulation of luciferase gene expression with antisense oligonucleotides: implications and applications in functional assay development,” Biochemistry, 37(18):6235-9, 1998.
Karin, Haslinger, Heguy, Dietlin and Cooke, “Metal-responsive elements act as positive modulators of human metallothionein-IIa enhancer activity,” Mol. Cell. Biol., 7:606, 1987.
Kashani-Sabet et al., “Reversal of the malignant phenotype by an anti-ras ribozyme,” Antisense Res. Dev., 2:3-15, 1992.
Katinka, Vasseur, Montreau, Yaniv and Blangy, “Polyoma DNA sequences involved in the control of viral gene expression in murine embryonal carcinoma cells,” Nature, 290:720, 1981.
Katinka, Yaniv, Vasseur and Blangy, “Expression of polyoma early functions in mouse embryonal carcinoma cells depends on sequence rearrangements in the beginning of the late region,” Cell, 20:393, 1980.
Kawamoto, Makino, Niw, Sugiyama, Kimura, Anemura, Nakata and Kakunaga, “Identification of the human β-actin enhancer and its binding factor,” Mol. Cell. Biol., 8:267, 1988.
Kiledjian, Su and Kadesch, “Identification and characterization of two functional domains within the murine heavy-chain enhancer,” Mol. Cell. Biol., 8:145, 1988.
Kim and Cech, “Three-dimensional model of the active site of the self-splicing rRNA precursor of Tetrahymena,” Proc. Natl. Acad. Sci. USA 84:8788-92, 1987.
Klamut, Gangopadyhay, Worton and Ray, “Molecular and functional analysis of the muscle-specific promoter region of the Duchenne muscular dystrophy gene,” Mol. Cell. Biol., 10:193, 1990.
Klein, Wolf, Wu and Sanford, “High-velocity microprojectiles for delivering nucleic acids into living cells. 1987,” Biotechnology, 24:384-86, 1992.
Koch, Benoist and Mathis, “Anatomy of a new B-cell-specific enhancer,” Mol. Cell. Biol., 9:303, 1989.
Kotin et al., “Site-specific integration by adeno-associated virus,” Proc. Natl. Acad. Sci. USA 87:2211-15, 1990.
Kriegler and Botchan, “A retrovirus LTR contains a new type of eukaryotic regulatory element,” In: Eukaryotic Viral Vectors, Gluzman, Ed., Cold Spring Harbor, Cold Spring Harbor Laboratory, NY, 1982.
Kriegler and Botchan, “Enhanced transformation by a simian virus 40 recombinant virus containing a Harvey murine sarcoma virus long terminal repeat,” Mol. Cell. Biol. 3:325, 1983.
Kriegler, Perez and Botchan, “Promoter substitution and enhancer augmentation increases the penetrance of the SV40 a gene to levels comparable to that of the Harvey murine sarcoma virus Ras gene in morphologic transformation,” In: Gene Expression, Hamer and Rosenberg, Eds., New York, Alan R. Liss, 1983.
Kriegler, Perez, Defay, Albert and Liu, “A novel form of TNF/cachectin is a cell-surface cytotoxix transmembrane protein: Ramifications for the complex physiology of TNF,” Cell, 53:45, 1988.
Kriegler, Perez, Hardy and Botchan, “Transformation mediated by the SV40 T antigens: separation of the overlapping SV40 early genes with a retroviral vector,” Cell, 38:483, 1984a.
Kriegler, Perez, Hardy and Botchan, “Viral integration and early gene expression both affect the efficiency of SV40 transformation of murine cells: Biochemical and biological characterization of an SV40 retrovirus,” In: Cancer Cells 2/Oncogenes and Viral Genes, Van de Woude, Levine, Topp and Watson, Eds., Cold Spring Harbor, Cold Spring Harbor Laboratory, 1984b.
Kuby, In: Immunology, 2nd Edition. W.H. Freeman & Company, New York, 1994.
Kuhl, De La Fuenta, Chaturvedi, Parinool, Ryals, Meyer and Weissman, “Reversible silencing of enhancers by sequences derived from the human IFN-α promoter,” Cell, 50:1057, 1987.
Kunz, Zimmerman, Heisig and Heinrich, “Identification of the promoter sequences involved in the Interleukin-6-dependent expression of the rat a-2-macroglobulin gene,” Nucl. Acids Res., 17:1121, 1989.
Kwoh, Davis, Whitfield, Chappelle, DiMichele and Gingeras, “Transcription-based amplification system and detection of amplified human immunodeficiency virus type 1 with a bead-based sandwich hybridization format,” Proc. Natl. Acad. Sci. USA, 86:1173-77, 1989.
Kyte and Doolittle, “A simple method for displaying the hydropathic character of a protein,” J. Mol. Biol., 157:105-32, 1982.
Larsen, Hamey and Moore, “Repression medaites cell-type-specific expression of the rat growth hormone gene,” Proc. Natl. Acad. Sci. USA., 83:8283, 1986.
Lasic, “Novel applications of liposomes,” Trends Biotechnol., 16:307-21, 1998.
Laspia, Rice and Mathews, “HIV-1 tat protein increases transcriptional initiation and stabilizes elongation,” Cell, 59:283, 1989.
Latimer, Berger and Baumann, “Highly conserved upstream regions of the α-antitrypsin gene in two mouse species govern liver-specific expression by different mechanisms,” Mol. Cell. Biol., 10:760, 1990.
Laughlin, Tratschin, Coon and Carter, “Cloning of infectious adeno-associated virus genomes in bacterial plasmids,” Gene, 23:65-73, 1983.
Lee et al., “Functional analysis of the steroid hormone control region of mouse mammary tumor virus,” Nucleic Acids Res., 12:4191-206, 1984.
Lee, Mulligan, Berg and Ringold, “Glucocorticoids regulate expression of dihydrofolate reductase cDNA in mouse mammary tumor virus chimaeric plasmids,” Nature, 294:228, 1981.
Levin et al., “Dysregulation of arcuate nucleus preproneuropeptide Y mRNA in diet-induced obese rats,” Am. J. Physiol., 272:R1365-70, 1997.
Levin et al., “Increased potency and binding of mazindol to putative brain anorectic receptors in obesity-prone rats,” Brain Res., 668(1-2):171-179, 1994.
Levinson, Khoury, VanDeWoude and Gruss, “Activation of SV40 genome by 72-base-pair tandem repeats of Moloney sarcoma virus,” Nature, 295:79, 1982.
L'Huillier, David and Bellamy, “Cytoplasmic delivery of ribozymes leads to efficient reduction in α-lactalbumin mRNA levels in C1271 mouse cells,” EMBO J, 11:4411-18, 1992.
Li et al., “Leptin receptor activation of SH2 domain containing protein tyrosine phophate 2 modulates Ob receptor signal transduction,” Proc. Natl. Acad. Sci. USA, 96:9677-82, 1999.
Lieber, Sandig, Sommer, Bahring and Strauss, “Stable high-level gene expression in mammalian cells by T7 phage RNA polymerase,” Methods Enzymol., 217:47-66, 1993.
Lin, Cross, Halden, Dragos, Toledano and Leonard, “Delineation of an enhancer like positive regulatory element in the interleukin-2 receptor α-chain gene,” Mol. Cell. Biol., 10:850, 1990.
Lisziewicz et al., “Inhibition of human immunodeficiency virus type 1 replication by regulated expression of a polymeric Tat activation response RNA decoy as a strategy for gene therapy in AIDS,” Proc. Natl. Acad. Sci. USA, 90:8000-04, 1993.
Lopez-Berestein et al., “Liposomal amphotericin B for the treatment of systemic fungal infections in patients with cancer: a preliminary study,” J. Infect. Dis., 2151:704, 1985a.
Lopez-Berestein et al., “Protective effect of liposomal-amphotericin B against C. albicans infection in mice,” Cancer Drug Delivery, 2:183, 1985b.
Luria, Gross, Horowitz and Givol, “Promoter ehancer elements in the rearranged a-chain gene of the human T-cell receptor,” EMBO J, 6:3307, 1987.
Lusby and Bems, “Mapping of the 5′ termini of two adeno-associated virus 2 RNAs in the left half of the genome,” J. Virol., 41:518-26, 1982.
Lusky and Botchan, “Transient replication of bovine papilloma virus Type 1 plasmids: cis and trans requirements,” Proc. Natl. Acad. Sci. U.S.A., 83:3609, 1986.
Lusky, Berg, Weiher and Botchan, “Bovine papilloma virus contains an activator of gene expression at the distal end of the early transcription unit,” Mol. Cell. Biol. 3:1108, 1983.
Majors and Varmus, “A small region of the mouse mammary tumor virus long terminal repeat confers glucocorticoid hormone regulation on a linked heterologous gene,” Proc. Natl. Acad. Sci. U.S.A., 80:5866, 1983.
Maloy et al.; In: Microbial Genetics, 2nd Edition, Jones and Barlett Publishers, Boston, Mass., 1994.
Maniatis et al., “Molecular Cloning: a Laboratory Manual,” Cold Spring Harbor Laboratory, Cold Spring Harbor, N.Y., 1982.
Margalit, “Liposome-mediated drug targeting in topical and regional therapies,” Crit. Rev. Ther. Drug Carrier Syst., 12:233-61, 1995.
McCowen et al., “Leptin signaling in the hypothalamus of normal rats in vivo,” Endocrinol., 139:4442-47, 1998.
McLaughlin et al., “Adeno-associated virus general transduction vectors: Analysis of proviral structures,” J. Virol., 62:1963-73, 1988.
McNeall, Sanchez, Gray, Chesterman and Sleigh, “Hyperinducible gene expression from a metallotionein promoter containing additional metal-responsive elements,” Gene, 76:81, 1989.
Michel and Westhof, “Modeling of the three-dimensional architecture of group I catalytic introns based on comparative sequence analysis,” J. Mol. Biol., 216:585-610, 1990.
Miksicek, Heber, Schmid, Danesch, Posseckert, Beato and Schutz, “Glucocorticoid responsiveness of the transcriptional enhancer of Moloney murine sarcoma virus,” Cell, 46:203, 1986.
Mordacq and Linzer, “Co-localization of elements required for phorbol ester stimulation and glucocorticoid repression of proliferin gene expression,” Genes and Dev., 3:760, 1989.
Moreau, Hen, Wasylyk, Everett, Gaub and Chambon, “The SV40 base-repair repeat has a striking effect on gene expression both in SV40 and other chimeric recombinants,” Nucl. Acids Res., 9:6047, 1981.
Mori and Fukatsu, “Anticonvulsant effect of DN-1417 a derivative of thyrotropin-releasing hormone and liposome-entrapped DN-1417 on amygdaloid-kindled rats,” Epilepsia, 33:994-1000, 1992.
Muesing, Smith and Capon, “Regulation of mRNA accumulation by a human immunodeficiency virus trans-activator protein,” Cell, 48:691, 1987.
Muller et al., “Efficient transfection and expression of heterologous genes in PC12 cells,” Cell, Biol., 9:221-29, 1990.
Muzyczka and McLaughlin, “Use of adeno-associated virus as a mamalian transduction vector,” In: Current Communications in Molecular Biology: Viral Vectors, Glzman and Hughes (eds.), Cold Spring Harbor Laboratory Press, Cold Spring Harbor, N.Y., pp. 39-44, 1988.
Narazaki et al., Proc. Natl. Acad. Sci. USA, 95:13130-34, 1998.
Ng, Gunning, Liu, Leavitt and Kedes, “Regulation of the human β-actin promoter by upstream and intron domains,” Nuc. Acids Res., 17:601, 1989.
Nicholson et al., EMBO J, 18:375-85, 1999.
Nicolau and Gersonde, “Incorporation of inositol hexaphosphate into intact red blood cells, I. fusion of effector-containing lipid vesicles with erythrocytes,” Naturwissenschaften (Germany), 66:563-66, 1979.
Nicolau and Sene, “Liposome-mediated DNA transfer in eukaryotic cells,” Biochem. Biophys. Acta, 721:185-190, 1982.
Ohara, Dort and Gilbert, “One-sided polymerase chain reaction: the amplification of cDNA,” Proc. Natl. Acad. Sci. USA, 86:5673-77, 1989.
Ohkawa, Yuyama and Taira, “Activities of HIV-RNA targeted ribozymes transcribed from a ‘shot-gun’ type ribozyme-trimming plasmid,” Nuc. Acids Symp. Ser., 27:15-6, 1992.
Ojwang, Hampel, Looney, Wong-Staal and Rappaport, “Inhibition of human immunodeficiency virus type 1 expression by a hairpin ribozyme,” Proc. Natl. Acad. Sci. USA., 89:10802-06, 1992.
Ondek, Sheppard and Herr, “Discrete elements within the SV40 enhancer region display different cell-specific enhancer activities,” EMBO J, 6:1017, 1987.
Ono, Hirose, Miyazaki, Yamamoto and Matsumoto, “Transgenic medaka fish bearing the mouse tyrosinase gene: expression and transmission of the transgene following electroporation of the orange-colored variant,” Pigment Cell Res., 10:168-75, 1997.
Ornitz, Hammer, Davison, Brinster and Palmiter, “Promoter and enhancer elements from the rat elastase I gene function independently of each other and of heterologous enhancers,” Mol. Cell. Biol., 7:3466, 1987.
Palmiter, Chen and Brinster, “Differential regulation of metallothionein-thymidine kinase fusion genes in transgenic mice and their offspring,” Cell, 29:701, 1982.
Parks, Melnick, Rongey and Mayor, “Physical assay and growth cycle studies of a defective adeno-satellite virus,” J. Virol., 1:171-80, 1967.
Pech, Rao, Robbins and Aaronson, “Functional identification of regulatory elements within the promoter region of platelet-derived growth factor 2,” Mol. Cell. Biol., 9:396, 1989.
Perez-Stable and Constantini, “Roles of fetal y-globin promoter elements and the adult β-globin 3′ enhancer in the stage-specific expression of globin genes,” Mol. Cell. Biol., 10:1116, 1990.
Perreault, Wu, Cousinequ, Ogilvie and Cedergren, “Mixed deoxyribo- and ribo-oligonucleotides with catalytic activity,” Nature, 344:565, 1990.
Perrotta and Been, “Cleavage of oligoribonucleotides by a ribozyme derived from the hepatitis delta virus RNA sequence,” Biochem., 31:16, 1992.
Philip et al., “Efficient and sustained gene expression in primary T lymphocytes and primary and cultured tumor cells mediated by adeno-associated virus plasmid DNA complexed to cationic liposomes,” Mol. Cell Biol., 4:2411-18, 1994.
Picard and Schaffner, “A Lymphocyte-specific enhancer in the mouse immunoglobulin kappa gene,” Nature, 307:83, 1984.
Pieken, Olsen, Benseler, Aurup and Eckstein, “Kinetic characterization of ribonuclease-resistant 2′-modified hammerhead ribozymes,” Science, 253:314, 1991.
Pikul et al., “In vitro killing of melanoma by liposome-delivered intracellular irradiation, Arch. Surg., 122:1417-20, 1987.
Pinkert, Ornitz, Brinster and Palmiter, “An albumin enhancer located 10 kb upstream functions along with its promoter to direct efficient, liver-specific expression in transgenic mice,” Genes and Dev., 1:268, 1987.
Pinto-Alphandary, Balland and Couvreur, “A new method to isolate polyalkylcyanoacrylate nanoparticle preparations,” J. Drug Target, 3:167-69, 1995.
Pinto-Sietsma and Paul, “Transgenic rats as models for hypertension,” J. Hum. Hypertens., 11:577-81, 1997.
Ponta, Kennedy, Skroch, Hynes and Groner, “Hormonal response region in the mouse mammary tumor virus long terminal repeat can be dissociated from the proviral pomoter and has enhancer properties,” Proc. Natl. Acad. Sci. U.S.A., 82:1020, 1985.
Porton, Zaller, Lieberson and Eckhardt, “Immunoglobulin heavy-chain enhancer is required to maintain transfected γ2A gene expression in a pre-B-cell line,” Mol Cell. Biol., 10:1076, 1990.
Potter et al., “Enhancer-dependent expression of human K immunoglobulin genes introduced into mouse pre-B lymphocytes by electroporation,” Proc. Natl. Acad. Sci. USA, 81:7161-65, 1984.
Prokop and Bajpai, “Recombinant DNA Technology I,” Conference on Progress in Recombinant DNA Technology Applications, Potosi, Mich., Jun. 3-8, 1990, Ann. N.Y. Acad. Sci., 646:1-383, 1991.
Queen and Baltimore, “Immunoglobulin gene transcription is activated by downstream sequence elements,” Cell, 35:741, 1983.
Quinn, Farina, Gardner, Krutzsch and Levens, “Multiple components are required for sequence recognition of the API site in the Gibbon ape leukemia virus enhancer,” Mol. Cell. Biol., 9:4713, 1989.
Quintanar-Guerrero, Allemann, Doelker and Fessi, “Preparation and characterization of nanocapsules from preformed polymers by a new process based on emulsification-diffusion techinque,” Phamr. Res., 15:1056-62, 1998.
Redondo, Hata, Brocklehurst and Krangel, “A T-cell-specific transcriptional enhancer within the human T-cell receptor γ locus,” Science, 247:1225, 1990.
Reinhold-Hurek and Shub, “Self-splicing introns in tRNA genes of widely divergent bacteria,” Nature, 357:173-76, 1992.
Reisman and Rotter, “Induced expression from the Moloney murine leukemia virus long terminal repeat during differentiation of human myeloid cells is mediated through its transcriptional enhancer,” Mol. Cell. Biol., 9:3571, 1989.
Rendahl et al., “Regulation of gene expression in vivo following transduction by two separate rAAV vectors,” Nat. Biotechnol., 16:757-61, 1998.
Renneisen, “Inhibition of expression of human immunodeficiency virus-1 in vitro by antibody-targeted liposomes containing antisense RNA to the env region,” J. Biol. Chem., 265:16337-42, 1990.
Resendez Jr., Wooden and Lee, “Identification of highly conserved regulatory domains and protein-binding sites in the promoters of the rat and human genes encoding the stress-inducible 78-kilodalton glucose-regulated protein,” Mol. Cell. Biol., 8:4579, 1988.
Ripe, Lorenzen, Brenner and Breindl, “Regulatory elements in the 5′ flanking region and the first intron contribute to transcriptional control of the mouse α-1-type collagen gene,” Mol. Cell. Biol., 9:2224, 1989.
Rippe et al., “DNA-mediated gene transfer into adult rat hepatocytes in primary culture,” Mol. Cell Biol., 10:689-95, 1990.
Rittling, Coutinho, Amarm and Kolbe, “AP-1/jun-binding sites mediate serum inducibility of the human vimentin promoter,” Nuc. Acids Res., 17:1619, 1989.
Rose and Koczot, “Adenovirus-associated virus multiplication. VII. Helper requirement for viral deoxyribonucleic acid and ribonucleic acid synthesis,” J. Virol., 10:1-8, 1972.
Rose, Bems, Hoggan and Koczot, “Evidence for a single-stranded adenovirus-associated virus genome: formation of a DNA density hybrid on release of viral DNA,” Proc. Natl. Acad. Sci. USA, 64:863-69, 1969.
Rosen, Sodroski and Haseltine, “The location of cis-acting regulatory sequences in the human T-cell lymphotropic virus type III (HTLV-111/LAV) long terminal repeat,” Cell, 41:813, 1988.
Rossi, Elkins, Zaia and Sullivan, “Ribozymes as anti-HIV-1 therapeutic agents: principles, applications, and problems,” AIDS Res. Hum. Retrovir., 8:183, 1992.
Sakai, Helms, Carlstedt-Duke, Gustafsson, Rottman and Yamamoto, “Hormone-mediated repression: A negative glucocorticoid-response element from the bovine prolactin gene,” Genes and Dev., 2:1144, 1988.
Sambrook et al., “Molecular Cloning: A Laboratory Manual,” Cold Spring Harbor Laboratory, Cold Spring Harbor, N.Y., 1989.
Samulski, Bems, Tan and Muzyczka, “Cloning of adeno-associated virus into pBR322: rescue of intact virus from the recombinant plasmid in human cells,” Proc. Natl. Acad. Sci. USA, 79:2077-80, 1982.
Samulski, Srivastava, Bems and Muzyczka, “Rescue of adeno-associated virus from recombinant plasmids: gene correction within the terminal repeats of AAV,” Cell, 33:135-43, 1983.
Sarver, Cantin, Chang, Zaia, Ladne, Stephens and Rossi, “Ribozymes as a potential anti-HIV-1 therapeutic agents,” Science, 247:1222-25, 1990.
Satake, Furukawa and Ito, “Biological activities of oligonucleotides spanning the F9 point mutation within the enhancer region of polyoma virus DNA,” J. Virology, 62:970, 1988.
Saville and Collins, “A site-specific self-cleavage reaction performed by a novel RNA in Neurospora mitochondria,” Cell, 61:685-96, 1990.
Saville and Collins, “RNA-mediated ligation of self-cleavage products of a Neurospora mitochondrial plasmid transcript,” Proc. Natl. Acad. Sci. USA, 88:8826-30, 1991.
Scanlon, Jiao, Funato, Wang, Tone, Rossi and Kashani-Sabet, “Ribozyme-mediated cleavage of c-fos mRNA reduces gene expression of DNA synthesis enzymes and metallothionein,” Proc. Natl. Acad. Sci. USA, 88:10591-95, 1991.
Scaringe, Francklyn and Usman, “Chemical synthesis of biologically active oligoribonucleotides using β-cyanoethyl protected ribonucleoside phosphoramidites,” Nuc. Acids Res., 18:5433-41, 1990.
Schaffner, Schirm, Muller-Baden, Wever and Schaffner, “Redundancy of information in enhancers as a principle of mammalian transcription control,” J. Mol. Biol., 201:81, 1988.
Sculier et al., “Pilot study of amphotericin B entrapped in sonicated liposomes in cancer patients with fungal infections,” J. Cancer Clin. Oncol., 24:527-38, 1988.
Searle, Stuart and Palmiter, “Building a metal-responsive promoter with synthetic regulatory elements,” Mol. Cell. Biol., 5:1480, 1985.
Segal, “Biochemical Calculations,” 2nd Edition, John Wiley & Sons, New York, 1976.
Shafron, Simpkins, Jebelli, Day and Meyer, “Reduced MK801 binding in neocortical neurons after AAV-mediated transfections with NMDA-R1 antisense cDNA,” Brain Res. 784:325-28, 1998.
Sharp and Marciniak, “HIV Tar: An RNA enhancer?,” Cell, 59:229, 1989.
Shaul and Ben-Levy, “Multiple nuclear proteins in liver cells are bound to Hepatitis B virus enhancer element and its upstream sequences,” EMBO J., 6:1913, 1987.
Sherman, Basta, Moore, Brown and Ting, “Class II box consensus sequences in the HLA-DRα gene: Transcriptional function and interaction with nuclear proteins,” Mol. Cell. Biol., 9:50, 1989.
Sleigh and Lockett, “SV40 enhancer activation during retinoic-acid-induced differentiation of F9 embryonal carcinoma cells,” J. EMBO, 4:3831, 1985.
Spalholz, Yang and Howley, “Transactivation of a bovine papilloma virus transcriptional regulatory element by the E2 gene product,” Cell, 42:183, 1985.
Spandau and Lee, “Trans-activation of viral enhancers by the Hepatitis B virus X protein,” J Virology, 62:427, 1988.
Spandidos and Wilkie, “Host-specificities of papilloma virus, Moloney murine sarcoma virus and simian virus 40 enhancer sequences,” EMBO J., 2:1193, 1983.
Srivastava, Lusby and Bems, “Nucleotide sequence and organization of the adeno-associated virus 2 genome,” J. Virol., 45:555-64, 1983.
Stephens and Hentschel, “The bovine papilloma virus genome and its uses as a eukaryotic vector,” Biochem. J, 248:1, 1987.
Stuart, Searle and Palmiter, “Identification of multiple metal regulatory elements in mouse metallothionein-I promoter by assaying synthetic sequences,” Nature, 317:828, 1985.
Sullivan and Peterlin, “Transcriptional enhancers in the HLA-DQ subregion,” Mol. Cell. Biol., 7:3315, 1987.
Suzuki, Shin, Fjuikura, Matsuzaki and Takata, “Direct gene transfer into rat liver cells by in vivo electroporation,” FEBS Lett., 425:436-40, 1998.
Swartzendruber and Lehman, “Neoplastic differentiation: Interaction of simian virus 40 and polyoma virus with murine teratocarcinoma cells,” J. Cell. Physiology, 85:179, 1975.
Taira, Nakagawa, Nishikawa and Furukawa, “Construction of a novel RNA-transcript-trimming plasmid which can be used both in vitro in place of run-off and (G)-free transcriptions and in vivo as multi-sequences transcription vectors,” Nucl. Acids Res., 19:5125-30, 1991.
Takakura, “Drug delivery systems in gene therapy,” Nippon Rinsho, 56:691-95, 1998.
Takebe, Seiki, Fujisawa, Hoy, Yokota, Arai, Yoshida and Arai, “SRα: promoter: An efficient and versatile mammalian cDNA expression system composed of the simian virus 40 early promoter and the R-U5 segment of human T-cell leukemia virus Type 1 long terminal repeat,” Mol. Cell. Biol., 8:466, 1988.
Tavernier, Gheysen, Duerinck, Can der heyden and fiers, “Deletion mapping of the inducible promoter of human IFN-β gene,” Nature, 301:634, 1983.
Taylor and Kingston, “E1a trans-activation of human HSP70 gene promoter substitution mutants is independent of the composition of upstream and TATA elements,” Mol. Cell. Biol., 10:176, 1990b.
Taylor and Kingston, “Factor substitution in a human HSP70 gene promoter: TATA-dependent and TATA-independent interactions,” Mol. Cell. Biol., 10:165, 1990a.
Taylor, Solomon, Weiner, Paucha, Bradley and Kingston, “Stimulation of the human heat-shock protein 70 promoter in vitro by simian virus 40 large T antigen,” J. Biol. Chem., 264:15160, 1989.
Thiesen, Bosze, Henry and Charnay, “A DNA element responsible for the different tissue specificities of friend and Moloney retroviral enhancers,” J. Virology, 62:614, 1988.
Tratschin, Miller and Carter, “Genetic analysis of adeno-associated virus: properties of deletion mutants constructed in vitro and evidence for an adeno-associated virus replication function,” J. Virol., 51:611-19, 1984a.
Tratschin, West, Sandbank and Carter, “A human parvovirus, adeno-associated virus, as a eucaryotic vector: transient expression and encapsidation of the procaryotic gene for chloramphenicol acetyltransferase,” Mol. Cell. Biol., 4:2072-81, 1984b.
Treisman, “Identification of a protein-binding site that mediates transcriptional response to the c-fos gene to serum factors,” Cell, 46:567-74, 1986.
Tronche, Rollier, Bach, Weiss and Yaniv, “The rat albumin promoter: Cooperation with upstream elements is required when binding of APF/HNF 1 to the proximal element is partially impaired by mutation or bacterial methylation,” Mol. Cell. Biol., 9:4759, 1989.
Tronche, Rollier, Herbomel, Bach, Cereghini, Weiss and Yaniv, “Anatomy of the rat albumin promoter,” Mol. Biol. Med., 7:173, 1990.
Trudel and Constantini, “A 3′ enhancer contributes to the stage-specific expression of the human β-globin gene,” Genes and Dev., 6:954, 1987.
Tur-Kaspa et al., “Use of electroporation to introduce biologically active foreign genes into primary rat hepatocytes,” Mol. Cell Biol., 6:716-18, 1986.
Tyndall, La Mantia, Thacker, Favaloro and Kamen, “A region of the polyoma virus genome between the replication origin and late protein-coding sequences is required in cis for both early gene expression and viral DNA replication,” Nuc. Acids. Res., 9:6231, 1981.
Usman and Cedergren, “Exploiting the chemical synthesis of RNA,” Trends in Biochem. Sci., 17:334, 1992.
Usman et al., J. Am. Chem. Soc., 109:7845-54, 1987.
Van Cott, Lubon, Russell, Butler, Gwazdauskas, Knight, Drohan and Velander, “Phenotypic and genotypic stability of multiple lines of transgenic pigs expressing recombinant human protein C,” Transgenic Res., 6:203-12, 1997.
Van Heek et al., “Diet-induced obese mice develop peripheral, but not central, resistance to leptin,” J. Clin. Invest., 99:385-90, 1997.
Vanbever, Fouchard, Jadoul, De Morre, Preat and Marty, “In vivo noninvasive evaluation of hairless rat skin after high-voltage pulse exposure,” Skin Parmacol. Appl. Skin Physiol., 11:23-34, 1998.
Vannice and Levinson, “Properties of the human Hepatitis B virus enhancer: Position effects and cell-type nonspecificity,” J. Virology, 62:1305, 1988.
Vasseur, Kress, Montreau and Blangy, “Isolation and characterization of polyoma virus mutants able to develop in multipotential murine embryonal carcinoma cells,” Proc. Natl. Acad. Sci. U.S.A., 77:1068, 1980.
Ventura, Wang, Ragot, Perricaudet and Saragosti, “Activation of HIV-specific ribozyme activity by self-cleavage,” Nucl. Acids Res., 21:3249-55, 1993.
Wagner, Zatloukal, Cotten, Kirlappos, Mechtler, Curiel and Bimstiel, “Coupling of adenovirus to transferrin-polylysine/DNA complexes greatly enhances receptor-mediated gene delivery and expression of transfected genes,” Proc. Natl. Acad. Sci. USA, 89:6099-103, 1992.
Walker, et al., “Strand displacement amplification—an isothermal, in vitro DNA amplification technique,” Nucleic Acids Res., 20:1691-96, 1992.
Wang and Calame, “SV40 enhancer-binding factors are required at the establishment but not the maintenance step of enhancer-dependent transcriptional activation,” Cell, 47:241, 1986.
Wang et al., Brain Res., 802:75-88, 1998.
Wang, et al., “NGF gene expression in dividing and non-dividing cells from AAV-derived constructs,” Neurochem Res., 23(5):779-86, 1998.
Weber, De Villiers and Schaffner, “An SV40 ‘enhancer trap’ incorporates exogenous enhancers or generates enhancers from its own sequences,” Cell, 36:983, 1984.
Weerasinghe, Liem, Asad, Read and Joshi, “Resistance to human immunodeficiency virus type 1 (HIV-1) infection in human CD4+ lymphocyte-derived cell lines conferred by using retroviral vectors expression an HIV-1 RNA-specific ribozyme,” J. Virol., 65:5531-34, 1991.
Weinberger, Jat and Sharp, “Localization of a repressive sequence contributing to B-cell specificity in the immunoglobulin heavy-chain enhancer,” Mol. Cell. Biol., 8:988, 1984.
Winoto and Baltimore, “αβ-lineage-specific expression of the a T-cell receptor gene by nearby silencers,” Cell, 59:649, 1989.
Wong and Neumann, “Electric field mediated gene transfer,” Biochim. Biophys. Res. Commun., 107:584-87, 1982.
Woolf, Melton and Jennings, “Specificity of antisense oligonucleotides in vivo,” Proc. Natl. Acad. Sci. USA, 89:7305-09, 1992.
Wu and Dean, “Functional significance of loops in the receptor binding domain of Bacillus thuringiensis CryIIIA delta-endotoxin,” J. Mol. Biol., 255:628-640, 1996.
Wu and Wu, “Evidence for targeted gene delivery to HepG2 hepatoma cells in vitro,” Biochemistry, 27:887-92, 1988.
Wu and Wu, “Receptor-mediated in vitro gene transfections by a soluble DNA carrier system,” J. Biol. Chem., 262:4429-32, 1987.
Yang et al., “In vivo and in vitro gene transfer to mammalian somatic cells by particle bombardment,” Proc. Natl. Acad. Sci. USA, 87:9568-72, 1990.
Yokosuka et al., “Inhibition of neuropeptide Y (NPY)-induced feeding and c-Fos response in magnocellular paraventricular nucleus by a NPY receptor anatonist: A site of NPY action,” Endocrinol., 140:4494-500, 1999.
Yu, Ojwang, Yamada, Hampel, Rapapport, Looney and Wong-Staal, “A hairpin ribozyme inhibits expression of diverse strains of human immunodeficiency virus type 1,” Proc. Natl. Acad. Sci. USA, 90:6340-44, 1993.
Yutzey, Kline and Konieczny, “An internal regulatory element controls troponin I gene expression,” Mol. Cell. Biol., 9:1397, 1989.
Zambaux, Bonneaux, Gref, Maincent, Dellacherie, Alonso, Labrude and Vigneron, “Influence of experimental paparmeters on the characteristics of poly(lactic acid) nanoparticles prepared by a double emulsion method,” J. Controlled Release, 50:31-40, 1998.
Zhou, Giordano, Durbin and McAllister, “Synthesis of functional mRNA in mammalian cells by bacteriophage T3 RNA polymerase,” Mol. Cell Biol., 10:4529-37, 1990.
zur Muhlen, Schwarz and Mehnert, “Solid lipid nanoparticles (SLN) for controlled drug delivery—drug release and release mechanism,” Eur. J. Pharm. Biopharm., 45:149-55, 1998.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A recombinant adeno-associated viral vector comprising a polynucleotide that comprises at least a first nucleic acid segment that encodes a mammalian neprilysin protein, peptide or polypeptide.

2. The recombinant adeno-associated viral vector of claim 1, wherein said vector further comprises at least one promoter operably linked to said nucleic acid segment.

3. The recombinant adeno-associated viral vector of claim 2, wherein said promoter is selected from the group consisting of a CMV promoter, a β-actin promoter, an EF 1 promoter, a U1a promoter, a Tet-inducible promoter, a VP16-LexA promoter, and a U1b promoter.

4. The recombinant adeno-associated viral vector of claim 2, wherein said vector further comprises an enhancer operably linked to said nucleic acid segment.

5. The recombinant adeno-associated viral vector of claim 4, wherein said enhancer comprises a CMV enhancer.

6. The recombinant adeno-associated viral vector of claim 4, wherein said enhancer comprises a cell- or tissue-specific enhancer.

7. The recombinant adeno-associated viral vector of claim 1, wherein said polynucleotide further comprises at least a first mammalian intron sequence.

8. The recombinant adeno-associated viral vector of claim 1, wherein said nucleic acid segment encodes a human neprilysin protein, peptide or polypeptide.

9. The recombinant adeno-associated viral vector of claim 1, wherein said nucleic acid segment encodes a biologically-active human neprilysin protein, peptide or polypeptide.

10. The recombinant adeno-associated viral vector of claim 1, comprised within an adeno-associated viral particle.

11. The recombinant adeno-associated viral vector of claim 1, comprised within an isolated mammalian host cell.

12. An adeno-associated viral particle or virion that comprises the recombinant adeno-associated viral vector of claim 1.

13. A plurality of recombinant adeno-associated viral particles, wherein at least one of said particles comprises the recombinant adeno-associated viral vector of claim 1.

14. An isolated host cell that comprises the recombinant adeno-associated viral vector of claim 1.

15. The isolated host cell of claim 14, wherein said cell is a mammalian host cell.

16. The isolated host cell of claim 15, wherein said cell is a human, primate, murine, feline, canine, porcine, ovine, bovine, equine, epine, caprine, or lupine host cell.

17. The isolated host cell of claim 14, wherein said host cell is a mammalian endothelial, vascular, epithelial, liver, lung, heart, pancreas, kidney, muscle, bone, neural, or brain cell.

18. A composition comprising the recombinant adeno-associated viral vector of claim 1.

19. The composition of claim 18, further comprising a pharmaceutical excipient, buffer, or diluent.

20. The composition of claim 19, formulated for administration to a mammal.

21. The composition of claim 18, further comprising a liposome, a lipid, a lipid complex, a microsphere, a microparticle, a nanosphere, or a nanoparticle.

22. A therapeutic or diagnostic kit that comprises: (a) the recombinant adeno-associated viral vector of claim 1; and (b) instructions for using said kit.

23. A method for providing a mammal with a therapeutically-effective amount of a biologically-active neprilysin peptide, polypeptide or protein, said method comprising introducing into suitable cells of said mammal an effective amount of an adeno-associated viral vector that comprises a nucleic acid segment that encodes a mammalian neprilysin peptide, polypeptide, or protein, wherein said pepetide, polypeptide or protein is expressed in at least one of said cells.

24. The method of claim 23, wherein said vector comprises at least one promoter operably linked to said nucleic acid segment.

25. The method of claim 23, wherein said vector comprises a nucleic acid segment that encodes a mammalian neprilysin polypeptide.

26. The method of claim 25, wherein said vector comprises a nucleic acid segment that encodes a mammalian neprilysin polypeptide that comprises an at least 35 contiguous amino acid sequence from any one of SEQ ID NO:1 to SEQ ID NO:10.

27. The method of claim 26, wherein said vector comprises a nucleic acid segment that encodes a mammalian neprilysin polypeptide that comprises an at least 55 contiguous amino acid sequence from any one of SEQ ID NO:1 to SEQ ID NO:10.

28. The method of claim 27, wherein said vector comprises a nucleic acid segment that encodes a mammalian neprilysin polypeptide that comprises an at least 75 contiguous amino acid sequence from any one of SEQ ID NO:1 to SEQ ID NO:10.

29. The method of claim 47, wherein said vector comprises a nucleic acid segment that encodes a mammalian neprilysin polypeptide that comprises an at least 95 contiguous amino acid sequence from any one of SEQ ID NO:1 to SEQ ID NO:10.

30. The method of claim 29, wherein said vector comprises a nucleic acid segment that encodes a mammalian neprilysin polypeptide that comprises the amino acid sequence of any one of SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, or SEQ ID NO:10.

31. The method of claim 23, wherein said mammal has an increased level of β-amyloid protein compared to that of a normal mammal.

32. The method of claim 23, wherein said mammal has a defect, deficiency, or substantial absence of biologically-active neprilysin protein compared to that of a normal mammal.

33. The method of claim 23, wherein a plurality of cells from said mammal are provided with said vector ex vivo or in vitro.

34. The method of claim 33, further comprising the additional step of introducing said plurality of cells into said mammal.

35. A method for preventing β-amyloid protein accumulation in neural cells of a mammal, said method comprising introducing into suitable cells of said mammal an amount of an adeno-associated viral vector or an adeno-associated viral particle that comprises said vector; wherein said vector comprises a nucleic acid segment that encodes a biologically-active mammalian neprilysin polypeptide, and wherein said polypeptide is expressed in said cell in an amount and for a time effective to prevent said β-amyloid protein accumulation in said neural cells of said mammal.

36. The method of claim 35, wherein said mammal has, is diagnosed with, or is at risk for developing Alzheimer's disease.

37. A method for treating neprilysin deficiency in a mammal, said method comprising the step of introducing into suitable cells of said mammal a therapeutically-effective amount of an adeno-associated viral vector; wherein said vector comprises a nucleic acid segment that encodes a biologically-active mammalian neprilysin polypeptide, and further wherein said polypeptide is expressed in said cell in an amount and for a time effective to treat said neprilysin deficiency in said mammal.

38. A method for decreasing the level of beta amyloid protein in a mammal, said method comprising introducing into suitable cells of said mammal an amount of an adeno-associated viral vector or an adeno-associated viral particle that comprises said vector; wherein said vector comprises a nucleic acid segment that encodes a biologically-active mammalian neprilysin polypeptide, and wherein said polypeptide is expressed in said cell in an amount and for a time effective to decrease the level of said beta amyloid protein in said mammal.


SEQUENCE LISTING (11 sequences)

SEQ ID NO: 1 - protein, 750 aa, Homo sapiens
MGKSESQMDITDINTPKPKKKQRWTRLEISLSVLVLLLTIIAVRMIALYATYDDGICKSSDCIKSAARLIQNMDATTEPCRDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMRLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 2 - protein, 750 aa, Rattus norvegicus
MGRSESQMDITDINAPKPKKKQRWTPLEISLSVLVLLLTIIAVTMIALYATYDDGICKSSDCIKSAARLIQNMDASAEPCTDFFKYACGGWLKRNVIPETSSRYSNFDILRDELEVILKDVLQEPKTEDIVAVQKAKTLYRSCINESAIDSRGGQPLLTLLPDIYGWPVASQNWEQTYGTSWTAEKSIAQLNSKYGKKVLINFFVGTDDKNSTQHIIHFDQPRLGLPSRDYYECTGIYKEACTAYVDFMISVARLIRQEQRLPIDENQLSLEMNKVMELEKEIANATTKPEDRNDPMLLYNKMTLAKLQNNFSLEINGKPFSWSNFTNEIMSTVNINIQNEEEVVVYAPEYLTKLKPILTKYSPRDLQNLMSWRFIMDLVSSLSRNYKESRNAFRKALYGTTSETATWRRCANYVNGNMENAVGRLYVEAAFAGESKHVVEDLIAQIREVFIQTLDDLTWMDAETKKKAEEKALAIKERIGYPDDIISNENKLNNEYLELNYKEEEYFENIIQNLKFSQSKQLKKLREKVDKDEWISGAAVVNAFYSSGRNQIVFPAGILQPPFFSARQSNSLNYGGIGMVIGHEITHGFDDNGRNFNKDGDLVDWWTQQSANNFKDQSQCMVYQYGNFTWDLAGGQHLNGINTLGENIADNGGIGQAYRAYQNYVKKNGEEKLLPGLDLNHKQLFFLNFAQVWCGTYRPEYAVNSIKTDVHSPGNFRIIGTLQNSAEFADAFHCRKNSYMNPERKCRVW

SEQ ID NO: 3 - protein, 750 aa, Mus musculus
  misc_feature (38)..(38): Xaa can be any naturally occurring amino acid
MGRSESQMDITDINAPKPKKKQRWTPLEISLSVLVLLXTIIAVTMIALYATYDDGICKSSDCIKSAARLIQNMDASVEPCTDFFKYACGGWLKRNVIPETSSRYSNFDILRDELEVILKDVLQEPKTEDIVAVQKAKTLYRSCINESAIDSRGGQPLLKLLPDIYGWPVASDNWDQTYGTSWTAEKSIAQLNSKYGKKVLINFFVGTDDKNSTQHIIHFDQPRLGLPSRGYYECTGIYKEACTAYVDFMISVARLIRQEQSLPIDENQLSLEMNKVMELEKEIANATTKPEDRNDPMLLYNKMTLAKLQNNFSLEVNGKSFSWSNFTNEIMSTVNINIQNEEEVVVYAPEYLTKLKPILTKYSPRDLQNLMSWRFIMDLVSSLSRNYKESRNAFRKALYGTTSETATWRRCANYVNGNMENAVGRLYVEAAFAGESKHVVEDLIAQIREVFIQTLDDLTWMDAETKKKAEEKALAIKERIGYPDDIISNENKLNNEYLELNYREDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSANNFKDQSQCMVYQYGNFSWDLAGGQHLNGINTLGENIADNGGIGQAYRAYQNYVKKNGEEKLLPGLDLNHKQLFFLNFAQVWCGTYRPEYAVNSIKTDVHSPGNFRIIGTLQNSAEFADAFHCRKNSYMNPERKCRVW

SEQ ID NO: 4 - protein, 750 aa, Oryctolagus cuniculus
MGRSESQMDITDINTPKPKKKQRWTPLEISLSVLVLLLTVIAVTMIALYATYDDGICKSSDCIKSAARLIQNMDATAEPCTDFFKYACGGWLKRNVIPETSSRYSNFDILRDELEVILKDVLQEPKTEDIVAVQKAKTLYRSCVNETAIDSRGGQPLLKLLPDVYGWPVATQNWEQTYGTSWSAEKSIAQLNSNYGKKVLINFFVGTDDKNSMNHIIHIDQPRLGLPSRDYYECTGIYKEACTAYVDFMIAVAKLIRQEEGLPIDENQISVEMNKVMELEKEIANATTKSEDRNDPMLLYNKMTLAQIQNNFSLEINGKPFSWSNFTNEIMSTVNINIPNEEDVVVYAPEYLIKLKPILTKYFPRDFQNLFSWRFIMDLVSSLSRTYKDSRNAFRKALYGTTSESATWRRCANYVNGNMENAVGRLYVEAAFAGESKHVVEDLIAQIREVFIQTLDDLTWMDAETKKKAEEKALAIKERIGYPDDIVSNDNKLNNEYLELNYKEDEYFENIIQNLKFSQSKQLKKLREKVDKDEWITGAAIVNAFYSSGRNQIVFPAGILQPPFFSAQQSNSLNYGGIGMVIGHEITHGFDDNGRNFNKDGDLVDWWTQQSANNFKEQSQCMVYQYGNFSWDLAGGQHLNGINTLGENIADNGGIGQAYRAYQNYVKKNGEEKLLPGIDLNHKQLFFLNFAQVWCGTYRPEYAVNSIKTDVHSPGNFRIIGSLQNSVEFSEAFQCPKNSYMNPEKKCRVW

SEQ ID NO: 5 - protein, 770 aa, Perca flavescens
MPIYIIDRKFPDTSGELIQPAAEAGDLRMMETNPPKSAKKPRWTSLEVGLTTIVSLLFIVIVALIILFATQKTDEICTTGDCTQSASRLIENMDDSVDPCDNFYQYACGGWLKKNIIPETSSRYSTFDILRDELEVILKGVLEKTDEGEATLSTRAKTLYKSCTNESLIELRGGAPLLDMLPDVFEWPVAVDNWETNYGKTWRLEDVIAKLNEKYGTQLLVNFFVGTDDRDSNSYIIHFDQQTNLGLLSRDYYACTGPYAEACRAYEKFMIDLAKLIRIDRGLNISETDIREEVKRVMDLERDIANATDTPEDRNNPVLLYNKMELGDLNANFTLEVESQVFDWSYFTAKIMDTVNISVPDTEKVINYSPNYYRRLNLILARYNKRDLQNYMVWRFAMNMVVGLSRSYRDTRKAFRKALSGTTSEAAVWRQCALYVNNNMDNAVGRLYVQEAFSEKSKELMEEMIKDIREVFISNLDDLTWMDAETKKAAEEKARAIRERIGYSDNIKDDKYLNNEYNDLAYSAEEYFENILQNLEYVQKKRLRKLRVKVNKEEWVTGAAVVNAFYSSSKNQIVFPAGILQPPFFSKGQAKSLNYGGIGMVIGHEITHGFDDNGRNYDKDGDLKDWWTPGSTDRFLDLSKCIVNQYGNFSWDLANGLHLNGNNTLGENIADNGGIRQAYQAYKNYVEKHGEEPSLPGINLSHNQLFFLNFAQVWCGTHRPEQAVNSIKVDVHSPGKFRVLGSLQNFPEFAKAFNCKKNSYMVPANICRVW

SEQ ID NO: 6 - protein, 750 aa, Gallus gallus
MGKSESQMDITEMNAPKPKKKLRWSGLEIGLTVVVILLAIVAITMIVLYATYDDGVCKTSDCIKSAARIIENMDTTAEPCNDFYQYACGGWLKRNVIPETSSRYSNFDILRDELEVVLKDVLDTPSSNDITAVQKAKTLYRSCINETTIDSRGGMPLISLLANLSEWPVATNNWESSYGAAWTAETAIAQLTSRYGKKVLINFFVGTDDKNSTAHIIHIDQPGLGLPSRDYYECTGAYQEACSAYVDFMISVAKLILQERNITFNETQIAEEMKRVMDLEKEIANATTKSEDRNDPLLLYNKMTLAQLQNNFSLEINHMAFNWSKFINNIMSTVQIDVENTEHVVVYDPEYLTKLKSILNKYTPRELQNYMIWRFVMDLVNSLSRNYKDTRNAFRKALYGTTSETAVWRRCANYVNGNMENAVGRLYVQEAFAGDSKHVVEEMIADIRGVFIETLDDLTWMDAETKKKAEQKATAIKERIGYPDEIMTDDSKLNSEYQELNYKEEEYFENIIQNLVFTQKKRLKKLREKVDKEEWISGAAVVNAFYSASRNQIVFPAGILQPPFFSASQPKSLNYGGIGMVIGHEITHGFDDNGRNFNENGDLVDWWTEESARNFKDLSQCMVYQYGNFSWDLAGGQQLSGINTLGENIADNGGVRQAYKAYENFVKKNGKEKLLPGLDMNHQQLFFLNFAQVWCGTYRPEYAVNSIKTDVHSPGKFRVIGSLQNSPEFSEAFSCTTKSYMDPAKKCRVW

SEQ ID NO: 7 - protein, 774 aa, Rattus norvegicus
MGKSESSVGMMERADNCGRRRLGFVECGLLVLLTLLLMGAIVTLGVFYSIGKQLPLLNSLLHVSRHERTVVKRVLRDSSQKSDICTTPSCVIAAARILQNMDQSKKPCDNFYQYACGGWLRHHVIPETNSRYSVFDILRDELEVILKGVLEDSSVQHRPAVEKAKTLYRSCMNQSVIEKRDSEPLLNVLDMIGGWPVAMDKWNETMGPKWELERQLAVLNSQFNRRVLIDLFIWNDDQNSSRHVIYIDQPTLGMPSREYYFKEDSHRVREAYLQFMTSVATMLRRDLNLPGETDLVQEEMAQVLHLETHLANATVPQEKRHDVTALYHRMGLEELQERFGLKGFNWTLFIQNVLSSVQVELLPNEEVVVYGIPYLENLEEIIDVFPAQTLQNYLVWRLVLDRIGSLSQRFKEARVDYRKALYGTTMEEVRWRECVSYVNSNMESAVGSLYIKRAFSKDSKSIVSELIEKIRSVFVDNLDELNWMDEESKKKAQEKALNIREQIGYPDYILEDNNRHLDEEYSSLTFSEDLYFENGLQNLKNNAQRSLKKLREKVDQNLWIIGAAVVNAFYSPNRNLIVFPAGILQPPFFSKDQPQALNFGGIGMVIGHEITHGFDDNGRNFDKNGNMLDWWSNFSARHFRQQSQCMIYQYSNFSWELADNQNVNGFSTLGENIADNGGVRQAYKAYLQWLAEGGRDQRLPGLNLTYAQLFFINYAQVWCGSYRPEFAIQSIKTDVHSPLKYRVLGSLQNLPGFSEAFHCPRGSPMHPMNRCRIW

SEQ ID NO: 8 - protein, 770 aa, Homo sapiens
MVESAGRAGQKRPGFLEGGLLLLLLLVTAALVALGVLYADRRGKQLPRLASRLCFLQEERTFVKRKPRGIPEAQEVSEVCTTPGCVIAAARILQNMDPTTEPCDDFYQFACGGWLRRHVIPETNSRYSIFDVLRDELEVILKAVLENSTAKDRPAVEKARTLYRSCMNQSVIEKRGSQPLLDILEVVGGWPVAMDRWNETVGLEWELERQLALMNSQFNRRVLIDLFIWNDDQNSSRHIIYIDQPTLGMPSREYYFNGGSNRKVREAYLQFMVSVATLLREDANLPRDSCLVQEDMVQVLELETQLAKATVPQEERHDVIALYHRMGLEELQSQFGLKGFNWTLFIQTVLSSVKIKLLPDEEVVVYGIPYLQNLENIIDTYSARTIQNYLVWRLVLDRIGSLSQRFKDTRVNYRKALFGTMVEEVRWRECVGYVNSNMENAVGSLYVREAFPGDSKSMVRELIDKVRTVFVETLDELGWMDEESKKKAQEKAMSIREQIGHPDYILEEMNRRLDEEYSNLNFSEDLYFENSLQNLKVGAQRSLRKLREKVDPNLWIIGAAVVNAFYSPNRNQIVFPAGILQPPFFSKEQPQALNFGGIGMVIGHEITHGFDDNGRNFDKNGNMMDWWSNFSTQHFREQSECMIYQYGNYSWDLADEQNVNGFNTLGENIADNGGVRQAYKAYLKWMAEGGKDQQLPGLDLTHEQLFFINYAQVWCGSYRPEFAIQSIKTDVHSPLKYRVLGSLQNLAAFADTFHCARGTPMHPKERCRVW

SEQ ID NO: 9 - protein, 786 aa, Canis familiaris
MGRSESQMDITDISTPRPKKQRWTSLEISLSVLVLLLTIIAVTMIALYATYDAARLIQNMDATAEPCTDFFKYACGGWLKRNVIPETSSRYSNFDILRDELEVVLKDVLQEPKTEDIVAVQKAKTLYRSCINESAIDSRGGQPLLSLLPDIYDWPVATDNWEQTYGTSWTAEKSIAQLNSKYGKKVILNFFVGTDDKNSVNHIIHIDQPRLGLPSRDYYVCTGIYEEGTTEVETLGSGKPLPPTRVSGPRGIFRPGLRDVLQGCVVGALAPSSQWKGVGMWDDISHPGNGLIRKEKGLLIDENQLSLEMNRVMELEKEIASATTKPEDRNDPMLLYNKMTLAQIQNNFTLEIDGKPFSWSNFTNEIMSTVNINIPNEEEVVVYAPEYLTKLKLILTKYSSRDLQNLMSWRFIMDLVSSLSRNYKESRNAFRKALYGTTSETATWRRCANYVNGNMENAVGRLYVEAAFAGESKHVVEDLITQIRAVFIQTLDDLTWMDAETKKKAEEKALAIKERIGYPDDIISNDSKLDNEYLELNYREDEYFENIIQNLKFSQNKQLKKLREKVDKDEWISGAAVVNAFYSSGRNQIVFPAGILQPPFFSALQSNSLNYGGIGMVIGHEITHGFDDNGRNFNKDGDLVDWWTQQSANNFKDQSQCMVYQYGNFSWDLAGGQHLNGINTLGENIADNGGIGQAYRAYQNYVKKNGEEKLLPGLDLNHKQLFFLNFAQVWCGTYRPEYAVNSIKTDVHSPGNFRIIGTLQNSPEFSEAFHCRKNSYMNPEKKCRVW

SEQ ID NO: 10 - protein, 770 aa, Homo sapiens; identical to SEQ ID NO: 8

SEQ ID NO: 11 - DNA, 58 bp, Artificial
  Synthetic Oligonucleotide
atgaagttatgggatgtcgtggctgtctgcctggtgctgctccacaccgcgtccgccg